SEQUENCE LISTING (66 sequences)

SEQ ID NO: 1 - DNA, 27 bp, Artificial Sequence
  5′ end of Oligonucleotide sequence
aacaccgtaatttctactcttgtagat

SEQ ID NO: 2 - DNA, 22 bp, Artificial Sequence
  3′ end of Oligonucleotide sequence
agcttggcgtaactagatcttg

SEQ ID NO: 3 - DNA, 12669 bp, Artificial Sequence
  Lenti_AsCpf1-Blast
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagcgtttaaacttaagcttggtaccgccaccatgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatgagaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggcctctggctgggacgtgaataaggagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgatcggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacaaaaggccggcggccacgaaaaaggccggccaggcaaaaaagaaaaagggatccggcgcaacaaacttctctctgctgaaacaagccggagatgtcgaagagaatcctggaccgatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagaattcgatatcaagcttatcggtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 4 - DNA, 12432 bp, Artificial Sequence
  Lenti_LbCpf1-Blast
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagcgtttaaacttaagcttggtaccgccaccatgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcacaaaaggccggcggccacgaaaaaggccggccaggcaaaaaagaaaaagggatccggcgcaacaaacttctctctgctgaaacaagccggagatgtcgaagagaatcctggaccgatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagaattcgatatcaagcttatcggtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 5 - DNA, 8329 bp, Artificial Sequence
  Lenti_crRNA-Puro
ccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgttgactatcgtctcgctactctaccacttgtacttcagcggtcagcttactcgacttaacgtgcacgtgacacgttctagaccgtacatgcttacatgggatgaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggggc

SEQ ID NO: 6 - DNA, 55 bp, Artificial Sequence
  Lenti_crRNA-Puro_clonning FP1
caccggagacgttgactatcgtctcgctactctaccacttgtacttcagcggtca

SEQ ID NO: 7 - DNA, 55 bp, Artificial Sequence
  Lenti_crRNA-Puro_clonning RP1
aagctgaccgctgaagtacaagtggtagagtagcgagacgatagtcaacgtctcc

SEQ ID NO: 8 - DNA, 60 bp, Artificial Sequence
  Lenti_crRNA-Puro_clonning FP1
gcttactcgacttaacgtgcacgtgacacgttctagaccgtacatgcttacatgggatga

SEQ ID NO: 9 - DNA, 60 bp, Artificial Sequence
  Lenti_crRNA-Puro_clonning RP2
agcttcatcccatgtaagcatgtacggtctagaacgtgtcacgtgcacgttaagtcgagt

SEQ ID NO: 10 - DNA, 60 bp, Artificial Sequence
  AsCpf1 oligo library amplification FP
atttcttggctttatatatcttgtggaaaggacgaaacaccgtaatttctactcttgtag

SEQ ID NO: 11 - DNA, 60 bp, Artificial Sequence
  LbCpf1 oligo library amplification FP
tttcttggctttatatatcttgtggaaaggacgaaacaccgtaatttctactaagtgtag

SEQ ID NO: 12 - DNA, 57 bp, Artificial Sequence
  As/LbCpf1 oligo library amplification RP
gagtaagctgaccgctgaagtacaagtggtagagtagagatctagttacgccaagct

SEQ ID NO: 13 - DNA, 55 bp, Artificial Sequence
  Targeted deep sequencing FP
acactctttccctacacgacgctcttccgatctcttgtggaaaggacgaaacacc

SEQ ID NO: 14 - DNA, 59 bp, Artificial Sequence
  Targeted deep sequencing RP
gtgactggagttcagacgtgtgctcttccgatctttgtggatgaatactgccatttgtc

SEQ ID NO: 15 - DNA, 29 bp, Artificial Sequence
  Illumina indexing FP-1
aatgatacggcgaccaccgagatctacac

SEQ ID NO: 16 - DNA, 20 bp, Artificial Sequence
  Illumina indexing FP-2
acactctttccctacacgac

SEQ ID NO: 17 - DNA, 24 bp, Artificial Sequence
  Illumina indexing RP-1
caagcagaagacggcatacgagat

SEQ ID NO: 18 - DNA, 21 bp, Artificial Sequence
  Illumina indexing RP-2
gtgactggagttcagacgtgt

SEQ ID NO: 19 - DNA, 23 bp, Artificial Sequence
  qPCR for WPRE FP
gatacgctgctttaatgcctttg

SEQ ID NO: 20 - DNA, 26 bp, Artificial Sequence
  qPCR for WPRE RP
gagacagcaaccaggatttatacaag

SEQ ID NO: 21 - DNA, 22 bp, Artificial Sequence
  qPCR for ALB FP
gctgtcatctcttgtgggctgt

SEQ ID NO: 22 - DNA, 21 bp, Artificial Sequence
  qPCR for ALB RP
actcatgggagctgctggttc

SEQ ID NO: 23 - DNA, 26 bp, Artificial Sequence
  Site for forward primer in oligonucleotide      sequence
tatcttgtggaaaggacgaaacaccg

SEQ ID NO: 24 - DNA, 29 bp, Artificial Sequence
  Site for reverse primer in oligonucleotide      sequence
gttttagagctagaaatagcaagttaaaa

SEQ ID NO: 25 - DNA, 55 bp, Artificial Sequence
  Forward primer for SpCas9 oligo library      amplification
ttgaaagtatttcgatttcttggctttatatatcttgtggaaaggacgaaacacc

SEQ ID NO: 26 - DNA, 56 bp, Artificial Sequence
  Reverse primer for SpCas9 oligo library      amplification
tttcaagttgataacggactagccttattttaacttgctatttctagctctaaaac

SEQ ID NO: 27 - DNA, 61 bp, Artificial Sequence
  Forward primer for targeted deep sequencing      (SpCas9)
acactctttccctacacgacgctcttccgatcttggactatcatatgcttaccgtaacttg

SEQ ID NO: 28 - DNA, 61 bp, Artificial Sequence
  Reverse primer for targeted deep sequencing      (SpCas9)
gtgactggagttcagacgtgtgctcttccgatcttttgtctcaagatctagttacgccaag

SEQ ID NO: 29 - DNA, 20 bp, Artificial Sequence
  Forward primer for endogenous target 1-5
ttgctgtggcagagccagcg

SEQ ID NO: 30 - DNA, 27 bp, Artificial Sequence
  Reverse primer for endogenous target 1-5
ttgcttcactttaatcctttcttgcag

SEQ ID NO: 31 - DNA, 24 bp, Artificial Sequence
  Forward primer for endogenous target 6-10
ctcctgcaagaaaggattaaagtg

SEQ ID NO: 32 - DNA, 28 bp, Artificial Sequence
  Reverse primer for endogenous target 6-10
acctacctaatagttacttcctgaaggg

SEQ ID NO: 33 - DNA, 28 bp, Artificial Sequence
  Forward primer for endogenous target 11-14
ctcgttctttccatcaaatagtgtggtg

SEQ ID NO: 34 - DNA, 28 bp, Artificial Sequence
  Reverse primer for endogenous target 11-14
ctgcagtaattgttactctgtgtcttcc

SEQ ID NO: 35 - DNA, 26 bp, Artificial Sequence
  Forward primer for endogenous target 15-17
ttgagctgacccataaatacaacagg

SEQ ID NO: 36 - DNA, 24 bp, Artificial Sequence
  Reverse primer for endogenous target 15-17
ccctcttaactggatcagcaacgg

SEQ ID NO: 37 - DNA, 22 bp, Artificial Sequence
  Forward primer for endogenous target 18
tggggtcgccattgtagttccc

SEQ ID NO: 38 - DNA, 26 bp, Artificial Sequence
  Reverse primer for endogenous target 18
gtcacaaagatcagcatcaggcatgg

SEQ ID NO: 39 - DNA, 20 bp, Artificial Sequence
  Forward primer for endogenous target 19-22
cgttcacctgggaggggaag

SEQ ID NO: 40 - DNA, 28 bp, Artificial Sequence
  Reverse primer for endogenous target 19-22
tctgcaaagaactttattccgagtaagc

SEQ ID NO: 41 - DNA, 28 bp, Artificial Sequence
  Forward primer for endogenous target 23-28
cccaaaagacatattcacccagaatccc

SEQ ID NO: 42 - DNA, 26 bp, Artificial Sequence
  Reverse primer for endogenous target 23-28
caacatcaaggtgtgggcagggctgc

SEQ ID NO: 43 - DNA, 21 bp, Artificial Sequence
  Forward primer for endogenous target 29-30
acctggagtctgcagagctgg

SEQ ID NO: 44 - DNA, 24 bp, Artificial Sequence
  Reverse primer for endogenous target 29-30
aagcggtaaacaaaggatagctgg

SEQ ID NO: 45 - DNA, 25 bp, Artificial Sequence
  Forward primer for endogenous target 31-35
ccatgggaaacgaatacaggtctcg

SEQ ID NO: 46 - DNA, 23 bp, Artificial Sequence
  Reverse primer for endogenous target 31-35
cttcagaagaaaaacctccactc

SEQ ID NO: 47 - DNA, 25 bp, Artificial Sequence
  Forward primer for endogenous target 36-37
aactgagaaacagccagagaggaag

SEQ ID NO: 48 - DNA, 23 bp, Artificial Sequence
  Reverse primer for endogenous target 36-37
catctgatgctgactcagagcgc

SEQ ID NO: 49 - DNA, 17 bp, Artificial Sequence
  Forward primer for endogenous target 38-42
gctgccaccccctgctc

SEQ ID NO: 50 - DNA, 26 bp, Artificial Sequence
  Reverse primer for endogenous target 38-42
atcagaatgaaaaatctcacccctcc

SEQ ID NO: 51 - DNA, 19 bp, Artificial Sequence
  Forward primer for endogenous target 43-46
gtctccgtgatgggggtgg

SEQ ID NO: 52 - DNA, 31 bp, Artificial Sequence
  Reverse primer for endogenous target 43-46
ctgccttgtaagactttaaatattctgctcc

SEQ ID NO: 53 - DNA, 27 bp, Artificial Sequence
  Forward primer for endogenous target 47-48
aagccatattcagttttagggaaaagc

SEQ ID NO: 54 - DNA, 26 bp, Artificial Sequence
  Reverse primer for endogenous target 47-48
atttccaagtaagctgcaaggaaagc

SEQ ID NO: 55 - DNA, 26 bp, Artificial Sequence
  Forward primer for endogenous target 49-52
aagtcttacaaggcagagtaaagatc

SEQ ID NO: 56 - DNA, 21 bp, Artificial Sequence
  Reverse primer for endogenous target 49-52
gcagggtaaaacaatcggacc

SEQ ID NO: 57 - DNA, 26 bp, Artificial Sequence
  Forward primer for endogenous target 53-57
caaccacctcagaagagccagattcc

SEQ ID NO: 58 - DNA, 26 bp, Artificial Sequence
  Reverse primer for endogenous target 53-57
ctctgtagttatttgagcaatgccac

SEQ ID NO: 59 - DNA, 27 bp, Artificial Sequence
  Forward primer for endogenous target 58-64
cagtgaatatacaggattggggttgtg

SEQ ID NO: 60 - DNA, 23 bp, Artificial Sequence
  Reverse primer for endogenous target 58-64
acaactggtaaggtgggcccagg

SEQ ID NO: 61 - DNA, 27 bp, Artificial Sequence
  Forward primer for endogenous target 65-72
caagcacaaacaaatcaggctaaatcc

SEQ ID NO: 62 - DNA, 23 bp, Artificial Sequence
  Reverse primer for endogenous target 65-72
ccctgagcttgggggagagttac

SEQ ID NO: 63 - DNA, 21 bp, Artificial Sequence
  Forward primer for endogenous target 73-78
tcctctggggaaagagtggcc

SEQ ID NO: 64 - DNA, 23 bp, Artificial Sequence
  Reverse primer for endogenous target 73-78
tgtggggtcgttcctgatgaaac

SEQ ID NO: 65 - DNA, 26 bp, Artificial Sequence
  Forward primer for endogenous target 79-82
aactggtttagctagtgcatacatgc

SEQ ID NO: 66 - DNA, 25 bp, Artificial Sequence
  Reverse primer for endogenous target 79-82
ggtgggagtttctgttacaggcaac

(19) United States
(12) Patent Application Publication
(10) Pub. No.: US 2019/0136211 A1
(43) Pub. Date: May 9, 2019

(54) Method For In Vivo High-Throughput Evaluating Of RNA-Guided Nuclease Activity

(71) Applicant: INDUSTRY-ACADEMIC COOPERATION FOUNDATION YONSEI UNIVERSITY, Seoul (KR)

(72) Inventors: Hyong Bum KIM, Seoul (KR); Hui Kwon KIM, Chungcheongbuk-do (KR); Myung Jae SONG, Seoul (KR)

(21) Appl. No.: 16/096,849

(22) PCT Filed: Apr. 28, 2017
(86) PCT No.: PCT/KR2017/004610

§ 371 Date: Oct. 26, 2018

(30) Foreign Application Priority Data
Apr. 28, 2016 (KR) 10-2016-0052365

(51) Int. Cl. C12N 9/22; G16B 30/20

(52) U.S. Cl. CPC C12N 9/22; C40B 50/06; C12Y 301/00; G16B 30/20

(57) ABSTRACT

The present invention relates to a method for evaluating the activity of an RNA-guided nuclease in a cell in a high-throughput manner, and specifically to a method for evaluating the activity of an RNA-guided nuclease from the indel frequency of a cell library including an isolated oligonucleotide that comprises a guide RNA-encoding nucleotide sequence and a target nucleotide sequence. The method for analyzing the characteristics of an RNA-guided nuclease using the guide RNA-target sequence pair library of the present invention enables the evaluation of the activity of the RNA-guided nuclease in vivo in a high-throughput manner, and thus, the method can be very effectively utilized in all of the fields where the RNA-guided nuclease is applied.

### FIELD

The present invention relates to a method for evaluating the activity of an RNA-guided nuclease in vivo, specifically in cells, in a high-throughput manner, and more specifically, to a method for evaluating the activity of an RNA-guided nuclease from the indel frequency of a cell library including an isolated oligonucleotide that includes a guide RNA-encoding nucleotide sequence and a target nucleotide sequence.

### BACKGROUND

RNA-guided nuclease derived from prokaryotic immunity system of type II clustered regularly interspaced short palindromic repeats and CRISPR-associated protein (CRISPR-Cas) provides a means for genome editing. In particular, studies have been actively conducted on techniques for editing genomes of cells and organs using single-guide RNA (sgRNA) and Cas9 protein (Cell, 2014, 157:1262-1278). In particular, studies for the prediction of sgRNA activity are being carried out in the CRISPR-Cas9 system (ACS Synth Biol., 2017, Feb. 10; Sci Rep, 2016, 6:30870, Nat Biotechnol, 34, 184-191), and studies are being conducted in China with regard to the use of CRISPR-Cas9 for the treatment of diseases by injecting cells, where genes encoding PD-1 are removed, by CRISPR-Cas9 (Nature, 2016, 539:479). Recently, Cpf1 protein (CRISPR derived from Prevotella and Francisella 1) was reported as another nuclease protein of class 2 CRISPR-Cas system (Cell, 2015, 163:759-771), and accordingly, the range of options for genome editing has been expanded. Cpf1 has various advantages in that it cuts in the form of a 5′ protrusion, has a shorter length of guide RNA, and has a longer distance between the seed sequence and the cut position. However, there is a lack of studies on the characteristics of Cpf1 in humans and other eukaryotic cells, and particularly in relation to target and off-target effects.

Although the activity and accuracy are very important in the application of RNA-guided nuclease to genome editing, a lot of time and efforts are required for the confirmation of the activity of targets and off-targets of RNA-guided nuclease. The accuracy of prediction with regard to the activity of targets and off-targets in silico is limited (Nat Biotechnol, 2014, 32:1262-1267), and there is a need for the characterization of nuclease through comprehensive in vivo experiments on RNA-guided nuclease activity so as to develop computer prediction models.

### SUMMARY

### Technical Problem

The present inventors have made efforts to develop a system that can evaluate the activity of RNA-guided nuclease in vivo conditions in a high-throughput manner, and as a result, have successfully developed a pair library system having guide RNA and a target sequence pair as major constituting elements thereby completing the present invention.

### Technical Solution

An object of the present invention is to provide a method for evaluating the activity of an RNA-guided nuclease, which includes: (a) performing sequence analysis using DNA obtained from a cell library, where an RNA-guided nuclease is introduced, which includes an oligonucleotide, containing a guide RNA-encoding nucleotide sequence and a target nucleotide sequence which the guide RNA targets; and (b) detecting the indel frequency of each guide RNA-target sequence pair from the data obtained from the sequence analysis.

Another object of the present invention is to provide a cell library including at least two kinds of cells, in which each cell includes an oligonucleotide containing a guide RNA-encoding nucleotide sequence and a target nucleotide sequence which the guide RNA targets.

Still another object of the present invention is to provide a vector containing an isolated oligonucleotide, which includes a guide RNA-encoding nucleotide sequence and a target nucleotide sequence which the guide RNA targets; and a vector library.

Still another object of the present invention is to provide an isolated oligonucleotide, which includes a guide RNA-encoding nucleotide sequence and a target nucleotide sequence which the guide RNA targets; and an oligonucleotide library.

Still another object of the present invention is to provide a method for constructing the oligonucleotide library, which includes: (a) setting a target nucleotide sequence, which is to be targeted with an RNA-guided nuclease; (b) designing a guide RNA-encoding nucleotide sequence, which forms a base pair with a complementary strand of the set target nucleotide sequence; (c) designing an oligonucleotide, which contains the target nucleotide sequence and a guide RNA that targets the same; and (d) repeating steps (a) to (c) at least once.

Still another object of the present invention is to provide an isolated guide RNA, which includes a sequence that is able to form a base pair with a complementary strand of a target nucleotide sequence that is adjacent to a proto-spacer-adjacent motif (PAM) sequence, that is, TTTV or CTTA.

Still another object of the present invention is to provide a composition for genome editing, which contains the isolated guide RNA or a nucleic acid encoding the same.

Still another object of the present invention is to provide a system for genome editing in a mammalian cell, which includes the isolated guide RNA, or a nucleic acid encoding the same; and a Cpf1 protein or a nucleic acid encoding the same.

Still another object of the present invention is to provide a method for genome editing with Cpf1 in a mammalian cell, which includes sequentially or simultaneously introducing the guide RNA or a nucleic acid encoding the same; and a Cpf1 protein or a nucleic acid encoding the same, into an isolated mammalian cell.

### Advantageous Effects

The method for evaluating the activity of an RNA-guided nuclease using the guide RNA-target sequence pair library of the present invention enables the evaluation of the activity of the RNA-guided nuclease in a cell (in vivo) in a high-throughput manner, and thus, the method can be very effectively utilized in all of the fields where the RNA-guided nuclease is applied.

### BRIEF DESCRIPTION OF THE DRAWINGS

FIG. 1 shows a schematic diagram illustrating oligonucleotides containing a pair of a target sequence and a guide RNA sequence for evaluating the activity of Cpf1.

FIG. 2 shows a schematic diagram illustrating the map of AsCpf1 lentivirus vector. Psi, packaging signal; RRE, rev response element, WPRE, posttranscriptional regulatory element of woodchuck hepatitis virus; U6, U6 pol III promoter; cPPT, central polypurine tract; EFS, elongation factor 1a short promoter; BlastR, blasticidin resistance gene.

FIG. 3 shows a schematic diagram illustrating the map of LbCpf1 lentivirus vector. Psi, packaging signal; RRE, rev response element, WPRE, posttranscriptional regulatory element of woodchuck hepatitis virus; U6, U6 pol III promoter; cPPT, central polypurine tract; EFS, elongation factor 1a short promoter; BlastR, blasticidin resistance gene.

FIG. 4 shows a schematic diagram of lentivirus vector, which includes backbone vector and a pair of a target sequence and guide RNA sequence, for the preparation of a plasmid library. Psi, packaging signal; RRE, rev response element; WPRE, posttranscriptional regulatory element of woodchuck hepatitis virus; cPPT, central polypurine tract; DR, direct repeat of Cpf1; GS, guide sequence of guide RNA; T, polyT; B, barcode; TS, target sequence; HS, homology sequence; EF1α, elongation factor 1 α promoter; PuroR, puromycin resistance gene.

FIG. 5 shows a schematic diagram briefly illustrating the entire process of a high-throughput analysis system using the pair library of the present invention.

FIG. 6 shows the relative copy number of each pair in an oligonucleotide pool, a plasmid library, and a cell library.

FIG. 7 shows the copy number of each pair in a plasmid library and a cell library normalized to the copy number of each pair in an oligonucleotide pool and a plasmid library.

FIG. 8 shows the relative copy number of each pair in a plasmid library and a cell library in the order of the copy number in an oligonucleotide pool.

FIG. 9 shows the relative copy number of each pair in a cell library in the order of the copy number in a plasmid library.

FIG. 10 shows the correlation between the pair copy number of a plasmid library and an oligonucleotide pool by evaluation through deep sequencing.

FIG. 11 shows the correlation between the pair copy number of a cell library and an oligonucleotide pool by evaluation through deep sequencing.

FIG. 12 shows the correlation between the pair copy number of a cell library and a plasmid library by evaluation through deep sequencing.

FIG. 13 shows a schematic diagram of the process for confirming the PAM sequences of AsCpf1 and LbCpf1.

FIG. 14 shows the indel frequency according to the potential PAM sequence of AsCpf1. The ANNNN sequence was experimented as a potential PAM sequence. For the purpose of brief representation, “A” was omitted.

FIG. 15 shows the indel frequency with regard to 4 kinds of TTTN PAM sequences of AsCpf1. Each error bar represents standard error of mean (SEM). *P<0.05, **P<0.01, ***P<0.001.

FIG. 16 shows the indel frequency according to the potential PAM sequence of LbCpf1. The ANNNN sequence was experimented as a potential PAM sequence. For the purpose of brief representation, “A” was omitted.

FIG. 17 shows the indel frequency with regard to 4 kinds of TTTN PAM sequences of LbCpf1. Each error bar represents standard error of mean (SEM). *P<0.05, **P<0.01, ***P<0.001.

FIG. 18 shows graphs illustrating the comparison results of PAM sequences by in vivo and in vitro analysis, in which a and b represent the results of in vitro analysis of the PAM sequence of (a)AsCpf1 and the PAM sequence of (b)LbCpf1, respectively; and c and d represent the results of in vivo analysis of the correlation between indel frequency and potential PAM sequences of (c)AsCpf1 and (d)LbCpf1, respectively.

FIG. 19 shows the indel frequency with regard to 4 kinds of NTTTA PAM sequences of AsCpf1 (left) and LbCpf1 (right). Each error bar represents standard error of mean (SEM). *P<0.05 ANOVA followed by Tukey's post hoc test.

FIG. 20 shows the comparison results with regard to the order of indel frequency between AsCpf1 and SpCas9 using forward or reverse target sequences. The correlation of the order of indel frequency with regard to forward target sequence (left) and reverse target sequence (right) of SpCas9 and AsCpf1 are shown. The 5′-GGG-3′ and 5′-TTTA-3′ sequences indicated in red were used as PAM sequences for SpCas9 and AsCpf1 target sequences, respectively. The order of activity with regard to the SpCas9 target sequence was referred to the literature (Nat Biotechnol, 2014, 32:1262-1267).

FIG. 21 shows a graph illustrating the nucleotide preference at each position of AsCpf1 target sequence with regard to the guide RNA with top 20% with high activity. The P-values were calculated by binomial distribution with baseline probability of 0.2 using 1,251 pairs of the guide RNA and target sequences from the literature (Nat Biotechnol, 2014, 32:1262-1267).

FIG. 22 shows a graph illustrating the relationship between GC contents of target sequences and indels observed, in which a, b, and c represent each group having statistically different indel frequency (P>0.05), and each error bar represents standard error of mean (SEM). *P<0.05, **P<0.01, ***P<0.001.

FIG. 23 shows a graph illustrating the average indel frequency according to time after delivery of Cpf1-expressing lentivirus vector in a cell library, in which each error bar represents standard error of mean (SEM). **P<0.01, ***P<0.001.

FIG. 24 shows the indel frequency at each target sequence on day 3, 5, and 31 after transduction of Cpf1-expressing lentivirus into a cell library.

FIG. 25 shows a schematic diagram illustrating experimental designs for the analysis of indel frequency according to nucleotide mismatch in guide RNA-encoding sequences and target sequences.

FIG. 26 shows the indel frequency according to the position of nucleotide mismatch in off-target sequences.

FIG. 27 shows a graph illustrating the indel frequency according to the guide RNA length in an off-target sequence with one nucleotide mismatch and an on-target sequence, which is normalized into indel frequency in an on-target sequence.

FIG. 28 shows a graph illustrating relative indel frequency according to the number of nucleotide mismatch in an off-target sequence.

FIG. 29 shows graphs illustrating the effect of the number of mismatch nucleotides according to a region within the on-target sequence, in an off-target indel frequency induced by Cpf1. The off-target indel frequency was normalized to indel frequency in an on-target sequence.

FIG. 30 shows graphs illustrating the effect of multiple-mismatch of nucleotides of a region within the on-target sequence, in an off-target indel frequency induced by Cpf1.

FIG. 31 shows a graph illustrating the effect of mismatch types with regard to the relative indel frequency in a seed region of an off-target sequence. **P<0.01.

FIG. 32 shows a graph illustrating the effect of mismatch types with regard to the relative indel frequency in a trunk region of an off-target sequence. **P<0.01.

FIG. 33 shows a graph illustrating the effect of mismatch types with regard to the relative indel frequency in a promiscuous region of an off-target sequence. **P<0.01.

FIG. 34 shows an illustration illustrating the concept of a high-throughput evaluation system in vivo using the pair library of the present invention. Conventionally, RNA-guided nuclease had been measured by an individual and difficult method (a small-scale system, top). The present invention enables high-throughput evaluation (a plant system, bottom), and thus provides a new method for easy evaluation of RNA-guided nuclease on a large-scale.

FIG. 35 shows a schematic diagram illustrating oligonucleotides for evaluation of Cas9 activity, containing a pair of a target sequence and a guide RNA sequence.

FIG. 36 shows a schematic diagram illustrating the map of Cas9 lentivirus vector.

FIG. 37 shows graphs illustrating the results of guide RNA activity measured using a guide RNA-target sequence pair library; and

FIG. 38 shows a graph illustrating the results of guide RNA activity measured using the pair library of the present invention.

FIG. 39 shows a schematic diagram illustrating the interaction between crRNA nuclease and the Thr16 in the Cpf1 WED domain at position 1. The hydroxyl side chain of the Thr16 residue within the WED domain exhibits a polar interaction with the N₂ of the guanine base (a blue dotted line within the red circle). The side chains of a different nucleobase (e.g., O₂ of thymine and uracil) can exhibit a polar interaction similar to that of the Thr16 residue. However, since the above moieties are not present in adenine, the side chains form an unstable binding with thymine present at position 1 of a target DNA strand located adjacent to the PAM motif, in the crRNA adenine ribonucleobase. There exists a complementary interaction between the crRNA ribonucleotide (guanine is indicated) and a target sequence nucleotide (cytocine at position 1 is indicated). The diagram was prepared based on the data of PDB 5643.

FIG. 40 shows a graph illustrating the correlation between indel frequencies in an endogenous target position and a corresponding introduced synthetic sequence, in which a scatter plot for the 82 analyzed endogenous regions is shown.

FIG. 41 shows a graph illustrating the correlation between indel frequencies in an endogenous target position and a corresponding introduced synthetic sequence, in which a scatter plot for top 25% DNase-sensitive regions among the 82 regions is shown.

FIG. 42 shows graphs illustrating the correlation between indel frequencies in an endogenous target position and an introduced sequence, in which scatter plots for each of the DNase-sensitive regions for (a) top 25% to 50% (b) top 50% to 75%, and (c) 75% to 100% are shown.

FIG. 43 shows a graph illustrating the correlation between indel frequencies in a biological replicate. Two different libraries (library A and library B) were prepared by independent lentivirus production and transduction. The two libraries were transfected with Cpf1-encoding plasmids, and after 4 days, the indel frequency was analyzed in the cell libraries.

FIG. 44 shows a graph illustrating the correlation between indel frequencies after the delivery of Cpf1 by two different delivery methods. The cell library was transfected with a Cpf1 plasmid or transduced with a Cpf1 lentivirus vector. After 4 days (transfection) or 5 days (transduction), the indel frequency of the cell library was analyzed.

FIG. 45 shows graphs illustrating the results of comparison of costs between the conventional method and the high-throughput manner evaluation method for evaluating Cpf1 activity in a target sequence. The costs of material(left) and labor(right) were compared. The cost was indicated in USD and the labor unit was indicated as the amount of maximum work that a skilled person can be performed. In a case where there was a break over one hour (e.g., cultivation time), it was not calculated as labor.

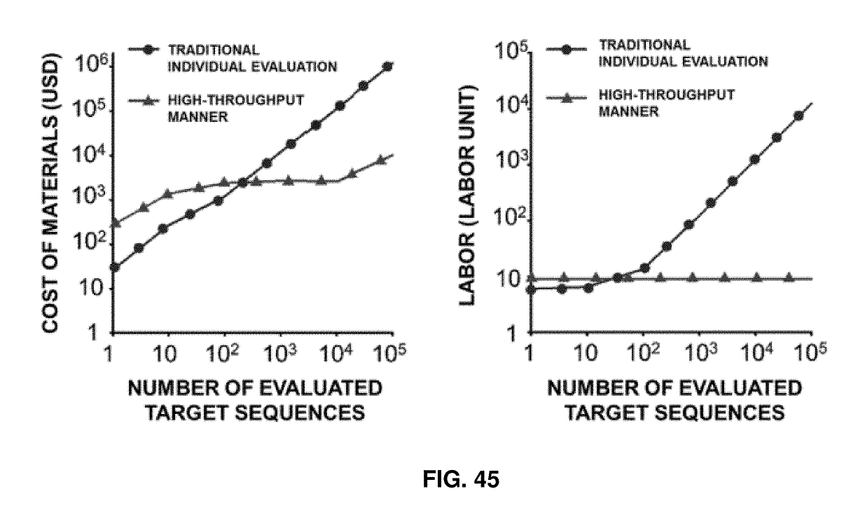

### DETAILED DESCRIPTION

Programmable nucleases are re widely used for genome editing of cells and individual subjects, and the technology employing the Programmable nucleases is a very useful technology that can be used for various purposes in life sciences, biotechnology, and medicine fields. In particular, recently, Cas9 which is RNA-guided nuclease derived from prokaryotic immunity system of type II CRISPR/Cas (clustered regularly interspaced repeat/CRISPR-associated), and Cpf1, etc., are attracting attention as its usefulness. However, for the utilization of the RNA-guided nucleases, it is important to design guide RNA with regard to its target sequence of these nucleases because on-target activity and off-target activity may vary depending on the sequence possessed by the guide RNA. In this regard, the present inventors have attempted to develop a method for evaluating the activity of RNA-guided nucleases in vivo in a high-throughput manner.

Herein below, exemplary embodiments of the present invention will be described in detail. Meanwhile, each of the explanations and exemplary embodiments disclosed herein can be applied to respective other explanations and exemplary embodiments. That is, all of the combinations of various factors disclosed herein belong to the scope of the present invention. Furthermore, the scope of the present invention should not be limited by the specific disclosure provided herein below.

To achieve the above objects, an aspect of the present invention provides a method for evaluating the activity of an RNA-guided nuclease, which includes: (a) performing sequence analysis using DNA obtained from a cell library, where an RNA-guided nuclease is introduced, including an oligonucleotide that includes a guide RNA-encoding nucleotide sequence and a target nucleotide sequence which the guide RNA targets; and (b) detecting the indel frequency of each guide RNA-target sequence pair from the data obtained from the sequence analysis. The present inventors have named the above method as “guide RNA-target sequence pair library analysis”, which refers to a method for evaluating the activity of an RNA-guided nuclease using a cell library where guide RNA-encoding nucleotide sequences and target nucleotide sequences are introduced thereinto as a pair.

In particular, the present inventors have confirmed that the activity of RNA-guided nucleases measured using the pair library has high correlation with the activity of RNA-guided nucleases acting on endogenous genes in a cell, and thereby, they have confirmed that the method for the evaluation of the RNA-guided nucleases of the present invention can not only be useful in vitro but also in vivo.

The technology of genome editing/gene editing is a technology that can introduce a target-directed modification to a nucleotide sequence of genome of animal/plant cells including humans, and it can also do knock-out or knock-in a particular gene or introduce modification to a non-coding DNA sequence which does not produce a protein. The method of the present invention can analyze on-target activity and off-target activity of RNA-guided nucleases used in the above technology of genome editing/gene editing in a high-throughput manner, and this can be effectively used for the development of a RNA-guided nuclease which only specifically acts on a target position.

As used herein, the term “RNA-guided nuclease” refers to a nuclease which is able to recognize a particular position on a target genome and cleave the same, and in particular, a nuclease having specificity by guide RNA. The RNA-guided nuclease may include Cas9 protein derived from CRISPR (i.e., a microorganism immune system), specifically CRISPR-associated protein 9 (Cas9), and Cpf1, etc., but RNA-guided nuclease is not limited thereto.

The RNA-guided nuclease may recognize a particular nucleotide sequence in the genome of animal/plant cells including human cells and cause a double strand break (DSB), and may form a nick (nicklase activity). The double strand break includes producing both blunt ends and cohesive ends by cleaving double strands of DNA. DSB is efficiently repaired by a mechanism of homologous recombination or non-homologous end-joining (NHEJ) in a cell, and the modification desired by a researcher may be introduced to a target site during this process. The RNA-guided nuclease may be artificial or manipulated non-naturally occurring.

As used herein, the term, “Cas protein” is a major protein constituting element of CRISPR/Cas system, and it is a protein that can act as an activated endonuclease or nickase. The Cas protein may form a complex with CRISPR RNA (crRNA) and trans-activating crRNA (tracrRNA), and thereby exhibit their activity.

The information on Cas protein or genes thereof may be obtained from a known database such as GenBank of National Center for Biotechnology Information (NCBI). Specifically, the Cas protein may be Cas9 protein. Additionally, the Cas protein may be derived from a microorganism of the genus Streptococcus, the genus Neisseria, the genus Pasteurella, the genus Francisella, and the genus Campylobacter, specifically, it derived from a microorganism of Streptococcus pyogenes, and more specifically Cas9 protein may be Cas9 protein derived from a microorganism of Streptococcus pyogenes, but is not limited thereto. However, the present invention is not limited to the examples described above, as long as it has the activity of the RNA-guided nuclease described above. In the present invention, the Cas protein may be a recombinant protein.

As used herein, the term “Cpf1” refers to a new nuclease of the CRISPR system, which is distinguished from the CRISPR/Cas system, and it was reported only recently (Cell, 2015, 163(3): 759-71). The Cpf1 is characterized in that it is a nuclease operated by single RNA, does not require tracrRNA, and has a relatively small size. Additionally, it is known that Cpf1 utilizes a thymine-rich protospacer-adjacent motif (PAM) sequence and generates a cohesive end by cleaving the double strand of DNA. The Cpf1 may be derived from a microorganism of the genus Candidatus Paceibacter, the genus Lachnospira, the genus Butyrivibrio, the genus Peregrinibacteria, the genus Acidominococcus, the genus Porphyromonas, the genus Prevotella, the genus Francisella, the genus Candidatus Methanoplasma, or the genus Eubacterium, but is not limited thereto. However, the present invention is not limited to the examples described above, as long as it has the activity of the RNA-guided nuclease described above. In the present invention, the Cpf1 protein may be a recombinant protein.

The above term “recombination”, for example, when it is used while mentioning cells, nucleic acids, proteins or vectors, etc., it means introduction of a heterologous nucleic acid or protein, or a change in native nucleic acid or protein, or a cell, nucleic acid, protein, or vector which is modified by a cell derived from a modified cell. Accordingly, for example, recombinant Cas9 or recombinant Cpf1 protein may be prepared by reconstituting the sequence encoding Cas9 protein or Cpf1 protein using the human codon table.

The Cas9 protein or Cpf1 protein may be in the form where the proteins are able to act in the nucleus, and may be in the form where they can easily be introduced into a cell. For example, the Cas9 protein or Cpf1 protein may be linked to a cell penetrating peptide or protein transduction domain. The protein transduction domain may be poly-arginine or a HIV-derived TAT protein, but is not limited thereto. With regard to the cell penetrating peptide or protein transduction domain, there are many kinds disclosed in the art, and thus those skilled in the art can apply various kinds, not limited to the above examples, to the present invention.

Additionally, any nucleic acid that encoding the Cas9 protein or Cpf1 protein can further include a nuclear localization signal (NLS) sequence. Accordingly, any expression cassette including nucleic acid encoding Cas9 protein or Cpf1 protein can include an NLS sequence, in addition to the control sequence (e.g., a promoter sequence, etc.) for the expression of the Cas9 protein or Cpf1 protein, but the sequence to be included is not limited thereto.

The Cas9 protein or Cpf1 protein may be linked to a tag which is useful for isolation and/or purification. For example, a small peptide tag (e.g., His tag, Flag tag, S tag, etc.), or a glutathione S-transferase (GST) tag, a maltose-binding protein (MBP) tag, etc. may be linked according to the purposes, but the tags are not limited thereto.

The present invention provides a method for analyzing the characteristics of the RNA-guided nuclease. Hereinafter, each step of the method will be described in detail. Meanwhile, as described above, it is apparent that the definitions and aspects of the terms described above are also applied to the following.

Step (a) is a step where deep sequencing is carried out using the DNA obtained from a cell library, which includes isolated oligonucleotide including guide RNA-encoding nucleotide sequences and target nucleotide sequences. The step is which data necessary for analysis are obtained from the cell population where various insertions and deletions (indels) occurred by the activity of on-target and off-target through acting the RNA-guided nuclease on various guide RNAs and target sequences.

Specifically, step (a) may be carried out, which includes:

(i) preparing an oligonucleotide library including a guide RNA-encoding nucleotide sequence and a target nucleotide sequence (i.e., a pair of a guide RNA sequence and a target nucleotide sequence),

(ii) preparing a vector library, specifically a virus vector library, using the oligonucleotide library and specifically preparing a vector library by preparing a vector for each oligonucleotide of the oligonucleotide library,

(iii) preparing a cell library using the vector library, specifically a virus vector library, and specifically, constructing a cell library by introducing each vector of the vector library into a cell, and

(iv) conducting sequence analysis (e.g., deep sequencing) using the DNA obtained from the cell library.

The cell library, where the DNA in step (iv) is obtained, may be one where RNA-guided nucleases are introduced into the cell library constructed in step (iii), and the activity of RNA-guided nuclease is induced by culturing the cells.

As used herein, the term “library” refers to a pool or population where two or more kinds of the same kind of material with different characteristics are included. Accordingly, the oligonucleotide library may be a pool including two or more kinds of oligonucleotides in which include a different nucleotide sequence (e.g., a guide RNA sequence, a PAM sequence) and/or a different target sequence; and the vector library (e.g., a virus vector library) may be a pool including two or more kinds of vectors in which include a different sequence or constituting element, for example, it may be a pool of vectors for each oligonucleotide of the oligonucleotide library, it may be a pool including two or more vectors which have a difference in the oligonucleotide constituting the corresponding vector. The cell library may be a pool of two or more kinds of cells with different characteristics, specifically a pool of cells including each different oligonucleotide for the purposes of the present invention, for example, a pool of cells including each different number of the introduced vectors and/or each different kinds of the introduced vectors, specifically cells including different kinds of the vectors. Since the present invention aims at evaluating the activity of RNA-guided nucleases using a cell library in high-throughput manner, the kinds of oligonucleotides, vectors (e.g., a virus vector), and cells of each library may be two or more kinds, and the upper limit of each library is not limited as long as the evaluation method is operated normally.

As used herein, the term “oligonucleotide” refers to a material where several to several hundred nucleotides are linked by phosphodiester bonds, and for the purposes of the present invention, the oligonucleotide may be double helix DNA. The oligonucleotide used in the present invention may have a length of 20 bp to 300 bp, specifically, 50 bp to 200 bp, and more specifically, 100 bp to 180 bp. In the present invention, the oligonucleotide includes a guide RNA-encoding nucleotide sequence and a target nucleotide sequence. Additionally, the oligonucleotide may include an additional sequence to which a primer can be bound for PCR amplification.

Specifically, in a single oligonucleotide, a guide RNA may be cis-acting on a target nucleotide sequence present adjacent to the same. That is, the guide RNA may be one which is designed so as to confirm whether the adjacent target nucleotide sequence has been cleaved.

The oligonucleotide may be introduced into a cell and integrated into the chromosome.

As used herein, the term “guide RNA” refers to a target DNA-specific RNA, and it may complementarily bind to all or part of a target sequence such that an RNA-guided nuclease cleaves the target sequence.

Conventionally, the guide RNA refers to a dual RNA which includes two RNAs (i.e., CRISPR RNA (crRNA) and trans-activating crRNA (tracrRNA)) as constituting elements; or a form which includes a first region including a complementary sequence in all or part of a sequence in the target DNA and a second region including a sequence interacting with an RNA-guided nuclease, but any form where the RNA-guided nuclease can have activity in a target sequence may be included without limitation in the scope of the present invention. In an embodiment, when the guide RNA is applied to Cpf1, the guide RNA may be crRNA, whereas when the guide RNA is applied to Cas, in particular Cas9, the guide RNA may be in the form of a dual RNA including crRNA and tracrRNA as constituting elements, or in the form of a single-chain guide RNA (sgRNA) where the major parts of crRNA and tracrRNA are fused. The sgRNA may include a part which has a sequence complementary to a sequence in the target DNA (this is called spacer region, target DNA recognition sequence, base pairing region, etc.) and a hairpin structure for the binding of Cas (especially Cas9 protein). More specifically, the sgRNA may include a part which has a sequence complementary to all or part of a sequence in the target DNA, a hairpin structure for the binding of Cas (especially Cas9 protein), and a terminator sequence. The structure described above may be one which is present sequentially in the 5′ to 3′ direction. However, the structure may not be limited thereto, but guide RNA in the form of any structure may be used in the present invention, as long as the guide RNA includes the major part of crRNA or all or part complementary to the target DNA.

The guide RNA, specifically crRNA or sgRNA, may include a sequence all or part of which is complementary to the sequence of the target DNA, an upstream part of crRNA or sgRNA, and specifically at least one additional nucleotide to the 5′ terminus of sgRNA or crRNA. The additional nucleotide may be guanine (G), but the nucleotide is not limited thereto.

Additionally, the guide RNA may include a scaffold sequence which helps the attachment of an RNA-guided nuclease.

As used herein, the term, “target nucleotide sequence or target sequence” refers to a nucleotide sequence which an RNA-guided nuclease is expected to target, and in the present invention, it further includes a target sequence to be analyzed by the method of guide RNA-a target nucleotide sequence pair library analysis of the present invention. In the present invention, a guide RNA and a target sequence are present in the form of a pair in each oligonucleotide and vector that constitutes the oligonucleotide library and the vector library, respectively. Therefore, the guide RNA present in one oligonucleotide or vector corresponds to its target sequence.

In the present invention, on-target activity (or on-target effect)/off-target activity (or off-target effect) and the target nucleotide sequence should be understood as completely distinct meanings.

The term “on-target activity” refers to activity, with regard to a sequence which is perfectly complementary to all or part of the sequence of guide RNA, which RNA-guided nuclease cleaves the sequence and further causes an indel on the cleaved region.

The term “off-target activity” refers to activity, with regard to a sequence which is not perfectly complementary to all or part of the sequence of guide RNA but part of the sequence mismatches, which RNA-guided nuclease cleaves the sequence and further causes an indel on the cleaved region. That is, the terms of “on-target activity” and “off-target activity” relate to a concept which is determined whether the sequence cleaved by the RNA-guided nuclease is perfectly complementary to all or part of the guide RNA sequence.

Meanwhile, the term “target sequence” as used herein refers to a sequence to be analyzed as to whether the activity of the RNA-guided nuclease occurred by the guide RNA present in the form of a pair is exhibited. That is, the target sequence can be determined by an operator during the process of design or preparation of each oligonucleotide that constitutes the oligonucleotide library of the present invention, and the operator can select according to the purpose of the embodiment in the designing step, the sequence from which on-target activity is expected and the sequence from which off-target activity is expected, with regard to the pair guide RNA and design the target sequence. The target sequence may include a protospacer-adjacent motif (PAM) sequence, which the RNA-guided nuclease recognizes, but is not limited thereto.

The design of an oligonucleotide may be freely conducted by those skilled in the art under the purpose of evaluating the activity of RNA-guided nucleases. For example, a pair may be comprised of sequences having on-target activity with regard to a particular guide RNA sequence, and also, a pair may be comprised of sequences having off-target activity with regard to the guide RNA sequence. For example, it is designed to a sequence which is perfectly complementary to guide RNA sequence, specifically the crRNA sequence, or it is designed to a sequence which is partially complementary such that part of the nucleotides mismatch.

Additionally, those skilled in the art may include additional constituting elements to oligonucleotides so as to perform the analysis of the guide RNA-target sequence pair library of the present invention. For example, the oligonucleotide may further include at least one selected from the group consisting of a direct repeat sequence, a poly T sequence, a barcode sequence, a constant region sequence, a promoter sequence, and a scaffold sequence, but the constituting elements are not limited thereto.

As described above, the oligonucleotide may be one consisting of a sequence of 100 to 200 nucleotides, but the oligonucleotide is not limited thereto, and may be appropriately adjusted by those skilled in the art according to the kinds, analysis purposes, etc. of the RNA-guided nuclease to be used.

Meanwhile, the oligonucleotide may be designed to include a target sequence and a guide RNA-encoding sequence in the 5′ to 3′ direction, and in contrast, may be designed to include guide RNA sequence and a target sequence in the 5′ to 3′ direction.

For example, the oligonucleotide may include a target sequence and a guide RNA-encoding sequence, specifically a target sequence, a barcode sequence, and a guide RNA-encoding sequence, and may be constructed in the following order, but the order is not particularly limited thereto.

The oligonucleotide may include a guide RNA-encoding sequence, a barcode sequence, and a target sequence in the 5′ to 3′ direction; specifically a guide RNA-encoding sequence, a barcode sequence, a PAM sequence, and a target sequence; a guide RNA-encoding sequence, a barcode sequence, target sequence, and a PAM sequence; a guide RNA-encoding sequence, a poly T sequence, a barcode sequence, a PAM sequence, and a target sequence; and a guide RNA-encoding sequence, a poly T sequence, a barcode sequence, a target sequence, and a PAM sequence.

More specifically, the oligonucleotide may include a direct repeat sequence, a guide RNA-encoding sequence, a barcode sequence, a PAM sequence, and a target sequence; a direct repeat sequence, a guide RNA-encoding sequence, a barcode sequence, a target sequence, and a PAM sequence; a direct repeat sequence, a guide RNA-encoding sequence, a barcode sequence, a PAM sequence, a target sequence, and a constant sequence; a direct repeat sequence, a guide RNA-encoding sequence, a barcode sequence, a target sequence, a PAM sequence, and a constant sequence, but the sequences are not particularly limited thereto.

Additionally, the oligonucleotide may further include a scaffold sequence which is adjacent to a guide RNA-encoding sequence and helps the binding of an RNA-guided nuclease.

For example, the oligonucleotide may include a scaffold sequence, a guide RNA-encoding sequence, a barcode sequence, a PAM sequence, and a target sequence, but the constituting elements are not particularly limited thereto.

Additionally, the oligonucleotide may include a promoter sequence at the 5′ end region for expression. In an embodiment of the present invention, a U6 promoter was used.

The oligonucleotide may include, in the 5′ to 3′ direction, a target sequence, a barcode sequence, and a guide RNA-encoding sequence; specifically may include a target sequence, a PAM sequence, a barcode sequence, and a guide RNA-encoding sequence; may include a PAM sequence, a target sequence, a barcode sequence, and a guide RNA-encoding sequence; may include a target sequence, a PAM sequence, a barcode sequence, a poly T sequence, and a guide RNA-encoding sequence; may include a PAM sequence, a target sequence, a barcode sequence, a poly T sequence, and a guide RNA-encoding sequence; more specifically may include a target sequence, a PAM sequence, a barcode sequence, a guide RNA-encoding sequence, and a direct repeat sequence; may include a PAM sequence, a target sequence, a barcode sequence, a guide RNA-encoding sequence, and a direct repeat sequence; may include a target sequence, a PAM sequence, a barcode sequence, a poly T sequence, a guide RNA-encoding sequence, and a direct repeat sequence; may include a PAM sequence, a target sequence, a barcode sequence, a poly T sequence, a guide RNA-encoding sequence, and a direct repeat sequence; may include a constant sequence, a target sequence, a PAM sequence, a barcode sequence, a poly T sequence, a guide RNA-encoding sequence, and a direct repeat sequence; may include a constant sequence, a PAM sequence, a target sequence, a barcode sequence, a poly T sequence, a guide RNA-encoding sequence, and a direct repeat sequence, but the constituting elements are not particularly limited thereto.

Additionally, the oligonucleotide may further include a scaffold sequence which is adjacent to a guide RNA-encoding sequence and helps the binding of the RNA-guided nuclease.

For example, the oligonucleotide may include a target sequence, a PAM sequence, a barcode sequence, a guide RNA-encoding sequence, and a scaffold sequence, but the constituting elements are not particularly limited thereto. Additionally, the oligonucleotide may include a promoter sequence at the 5′ end region for expression.

Additionally, as described above, the oligonucleotide may further include a primer attachment sequence at the 5′ end and 3′ end for PCR amplification in addition to the constituting elements described above, but the constituting elements are not particularly limited thereto.

The target sequence may have a length of 10 bp to 100 bp, specifically 20 bp to 50 bp, more specifically 23 bp to 34 bp, but the length is not particularly limited thereto.

Additionally, the guide RNA-encoding sequence may have a length of 10 bp to 100 bp, specifically 15 bp to 50 bp, and more specifically 20 bp to 30 bp, but the length is not particularly limited thereto.

Additionally, the barcode sequence refers to a nucleotide sequence for the recognition of each oligonucleotide. In the present invention, the barcode sequence may not include two or more of repeated nucleotides (i.e., AA, TT, CC, and GG), but the barcode sequence is not particularly limited as long as it is designed so as to recognize each oligonucleotide. In multiple oligonucleotides, the barcode sequence may be designed such that at least two nucleotides are different so as to distinguish each oligonucleotide. The barcode sequence may have a length of 5 bp to 50 bp, but the length is not particularly limited thereto.

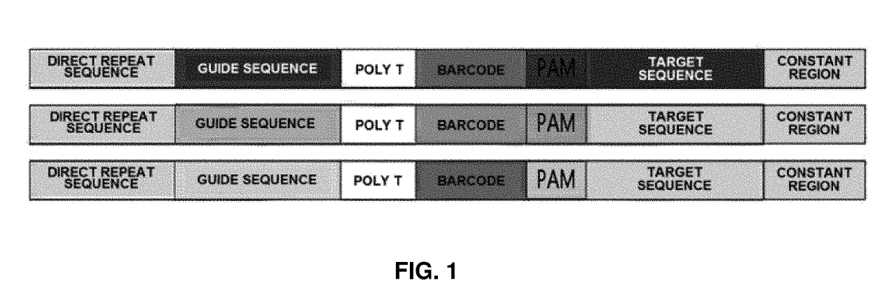

In a specific embodiment of the present invention, with regard to Acidaminococcus-derived Cpf1 (AsCpf1) and Lachnospiraceae-derived Cpf1 (LbCpf1), pair oligonucleotides were synthesized from 8,327 species and 3,634 species, respectively, by varying the guide RNA and/or target sequence, and thereby an oligonucleotide library including total 11,961 species of guide RNA-target sequence pair oligonucleotide were prepared. Each oligonucleotide constituting the oligonucleotide library had a total length of 122 bp to 130 bp nucleotides, and includes a mutually-different pair of a guide RNA-encoding sequence and a target nucleotide sequence, and the specific constitution is shown in FIG. 1.

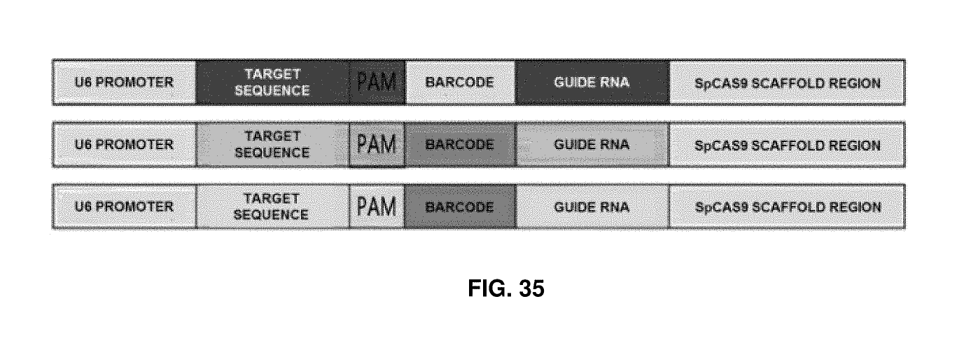

Additionally, in another embodiment of the present invention, with regard to Streptococcus pyogenes-derived Cas9 (SpCas9), 89,592 oligonucleotides were synthesized and thereby an oligonucleotide library including oligonucleotides of guide RNA-target sequence pairs were prepared. The oligonucleotide had a total length of 120 nucleotides and includes a guide RNA-encoding sequence (guide sequence) and a target sequence (FIG. 35).

Next, a vector library (e.g., a virus vector) can be prepared using the oligonucleotide library.

One of the advantages of the method for evaluating the activity of RNA-guided nucleases using the guide RNA-target sequence pair of the present invention lies in that the pair is introduced into a cell using a virus. Since the guide RNA corresponding to a target sequence is introduced into a cell in the form of a pair, the effects that may occur due to the deviation in copy number in an oligonucleotide library, vector library, and cell library can be minimized, and can be integrated into the genomic DNA through a virus, it is possible to perform analysis of the activity of on-target and off-target according to time unlike the analysis method by transient expression, and furthermore, the effects caused by epigenetic factors can be relatively minimized. When the vector is a virus, a virus library is introduced into a cell and virus can be produced therefrom and obtained, and cells can be infected using the same. This process can be appropriately performed by those skilled in the art using a method known in the art.

In the present invention, the vector may include oligonucleotides where each oligonucleotide includes a guide RNA-encoding nucleotide sequence and a target nucleotide sequence. The vector may be a virus vector or plasmid vector, and the virus vector may specifically be a lentivirus vector, retrovirus vector, etc., but the vectors are not limited thereto, and those skilled in the art can freely use any known vector that can achieve the objects of the present invention.

The vector refers to a mediator that can deliver the oligonucleotide to a cell, for example, a genetic construct. Specifically, when the vector is present in cells of an individual subject, it may include an insert, that is, an insert where an essential control element is operably linked thereto such that an oligonucleotide can be expressed.

The vector may be prepared and purified using a standard recombinant DNA technology. The kinds of the vector may not be particularly limited as long as the vector can act in the target cells (e.g., eukaryotes, prokaryotes, etc.). The vector may include a promoter, an initiation codon, and a termination codon terminator. In addition, the vector may appropriately include DNA encoding a signal peptide, and/or an enhancer sequence, and/or an untranslated region in the 5′ and 3′ sites of a gene, and/or a selective marker region, and/or a replicable unit, etc.

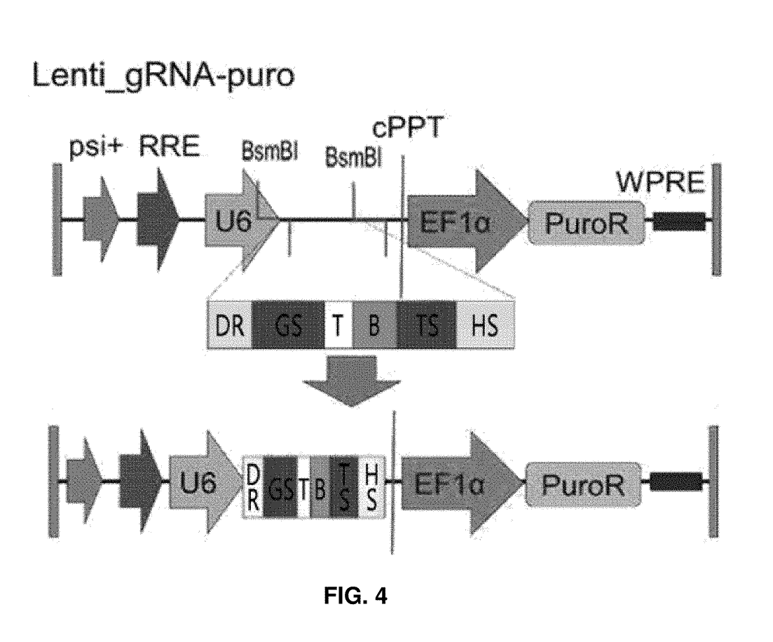

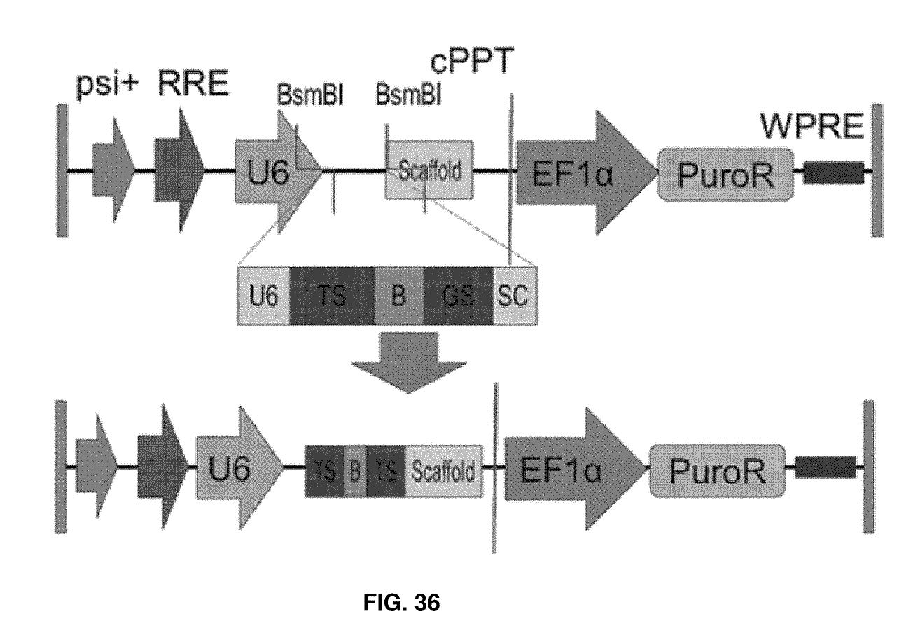

In a specific embodiment of the present invention, a lentivirus vector library was prepared by cloning each oligonucleotide of the oligonucleotide library into the lentivirus vector (FIGS. 4 and 36), and the same was expressed in cells and thereby the virus was obtained.

The next step is to prepare a cell library by introducing the vector into a target cell. Specifically, the method of delivering the vector to a cell for the preparation of a library can be achieved by various methods known in the art. These methods may include, for example, calcium phosphate-DNA co-precipitation method, a DEAE-dextran-mediated transfection method, polybrene-mediated transfection method, electroporation, microinjection, liposome fusion method, Lipofectamine® and protoplast fusion method, etc. which are known in the art. Additionally, when a virus vector is used, the target product (i.e., the vector) may be delivered using virus particles having the infection as a means. Additionally, the vector may be introduced into a cell by gene bombardment, etc.

The introduced vector may be present as a vector itself in a cell or may be integrated into the chromosome, but the vector state is not particularly limited thereto.

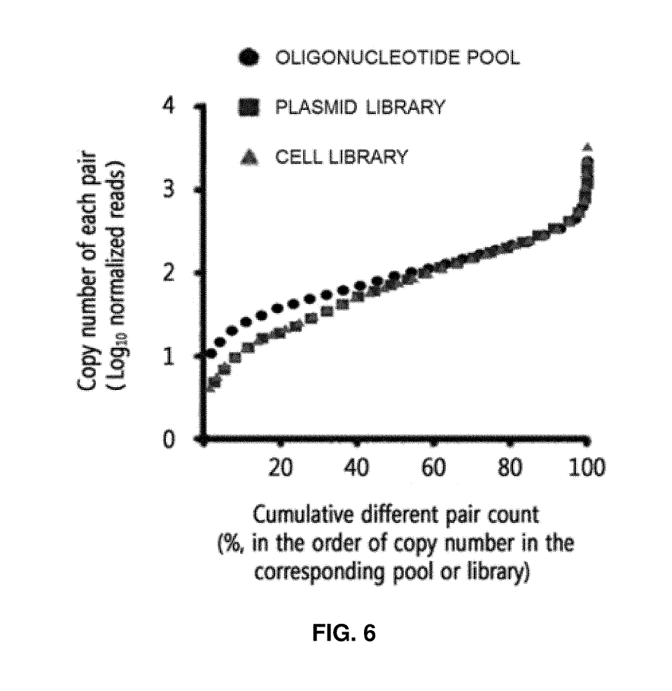

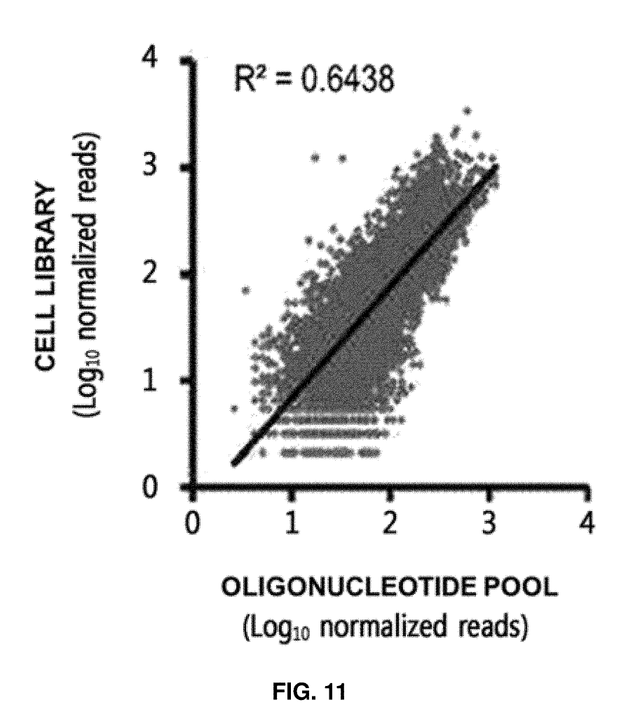

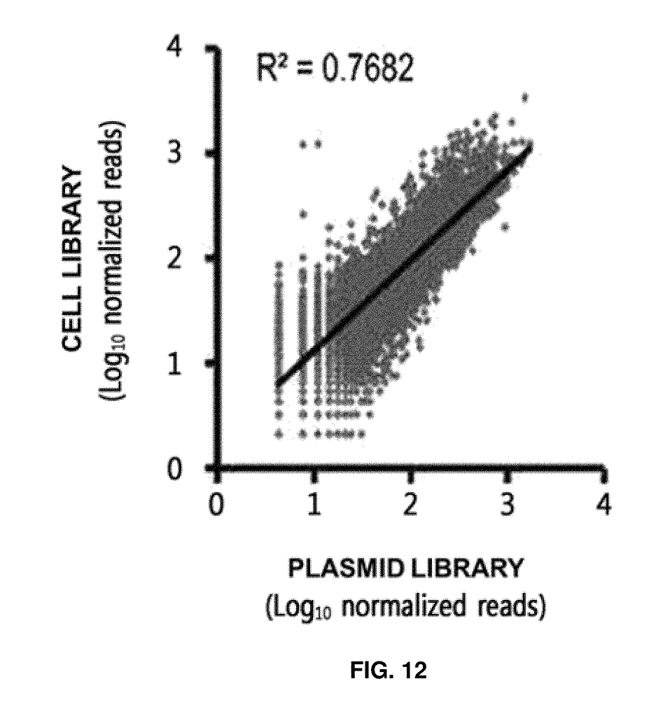

The cell library prepared in the present invention refers to a cell population in which oligonucleotides containing a guide RNA-target sequence are introduced. In particular, each cell may be those where the vector was introduced, and specifically the vector was introduced such that the kinds and/or number of the virus were different. However, the analysis method of the present invention is performed using all of the cell library, and the guide RNA-encoding nucleotide sequence and the target sequence are introduced in the form of a pair, and thus the method is not significantly affected by efficiency of cell infection, deviation in the copy number of oligonucleotides, etc. (FIGS. 6 to 12) and each pair-dependent interpretation is possible.

An RNA-guided nuclease may be further introduced so as to induce indel to the constructed cell library.

The nuclease may differently exhibit the degree of its activity according to the kinds and/or number of the guide RNA-target sequence pair. The RNA-guided nuclease may be delivered to a cell through a plasmid vector or virus vector, and may be delivered to a cell as an RNA-guided nuclease protein itself, but the introduction method is not particularly limited as long as the RNA-guided nuclease can exhibit its activity in cells. In an embodiment, the RNA-guided nuclease may be delivered in a form where it is linked to a protein transduction domain (e.g., a Cas protein, a Cpf1 protein, etc.), but the form is not limited thereto. As the protein transduction domain, various kinds known in the art may be used, and poly-arginine or a HIV-derived TAT protein may be used as described above, but is not particularly limited thereto.

Additionally, the kinds of cells into which the vector can be introduced may be appropriately selected by those skilled in the art according to the kinds of the vector and/or kinds of the target cells, for example, bacterial cells (e.g., E. coli, Streptomyces, Salmonella typhimurium, etc.); yeast cells; fungal cells (e.g., Pichia pastoris, etc.); insect cells (e.g., Drosophila, Spodoptera frugiperda (Sf9), etc.); animal cells (e.g., Chinese hamster ovary cells (CHO), SP2/0 (mouse myeloma), human lymphoblastoid, COS, NSO (mouse myeloma), 293T, bow melanoma cells, HT-1080, baby hamster kidney cells (BHK), human embryonic kidney cells (HEK), PERC.6 (human retinal cells), etc.); or plant cells.

In the cell library, the activity of nuclease may appear by the introduced guide RNA-target sequence pair oligonucleotide and an RNA-guided nuclease. That is, with regard to the introduced target sequence, a DNA cleavage by an RNA-guided nuclease may occur, and an indel may occur accordingly. As used herein, the term “indel” collectively refers to modification where, in a nucleotide sequence of DNA, part of the nucleotide is inserted or deleted. The indel may be one which, when an RNA-guided nuclease cleaves double-stranded DNA as described above, is introduced to a target sequence during the process while repair is conducted by a mechanism of homologous recombination or non-homologous end-joining (NHEJ).

Additionally, the method of the present invention may include obtaining a DNA sequence from the cell where the activity of the introduced RNA-guided nuclease is exhibited. The obtaining of DNA may be carried out using various DNA isolation methods known in the art.

Since it is expected that each cell constituting the cell library undergoes the occurrence of an indel in an introduced target sequence, the relevant data can be obtained by performing sequence analysis for the nucleotides of the target sequence (e.g., deep sequencing or RNA-seq).

Since the analysis method of the present invention using a guide RNA-target sequence pair library is performed in vivo, reliable analysis results can be obtained without artifacts compared to other analysis methods in vitro.

Accordingly, step (b) is a step of obtaining the indel frequency of each guide RNA-target sequence pair from the data obtained through the sequence analysis.

As described above, each indel may occur in a manner dependent on each guide RNA-target sequence pair, and accordingly, the indel frequency may be evaluated as the degree of activity of RNA-guided nuclease by the guide RNA-target sequence pair.

Each pair can be distinguished by inserting a particular sequence, to each oligonucleotide constituting the oligonucleotide library, which is able to distinguish the oligonucleotide, and thus it is possible to perform analysis by classifying the data based on the distinguished sequence in the step of data analysis. In an embodiment of the present invention, each oligonucleotide was prepared to include a barcode sequence which does not include any repeat of two or more nucleotides (i.e., AA, CC, TT, and GG) and include at least two mutually-different nucleotides.

The pair library of the present invention provides a method for evaluating the activity of RNA-guided nucleases with improved accuracy and predictability by having high correlation with the activity of the RNA-guided nucleases that act on the endogenous genes in vivo.

In a specific embodiment of the present invention, it was confirmed that the activity of programmable nucleases measured through libraries were highly correlated with the activity of the programmable nucleases which actually act on endogenous genes in vivo.

Additionally, the pair library of the present invention has an advantage in that it enables the evaluation of the activity of RNA-guided nucleases with high accuracy.

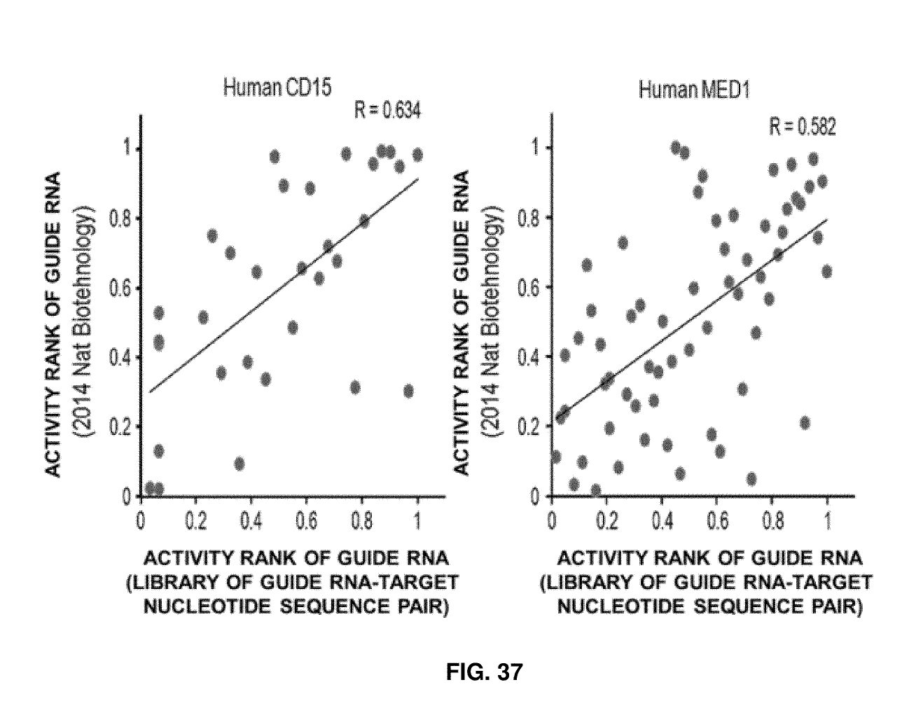

Specifically, in a specific embodiment of the present invention, the accuracy of a pair library was evaluated by comparing the activity ranking of the guide RNAs of the human CD15 gene and human MED1 gene with the activity ranking of the guide RNA disclosed previously (Nat Biotechnol, 2014, 32:1262-1267, Nat Biotechnol, 2016, 34:184-191). As a result, both guide RNAs for human CD15 and human MED1 gene showed high Spearman correlation coefficients, and thus it was confirmed that these guide RNAs have high correlation with the activity ranking of the known guide RNA (FIG. 37)

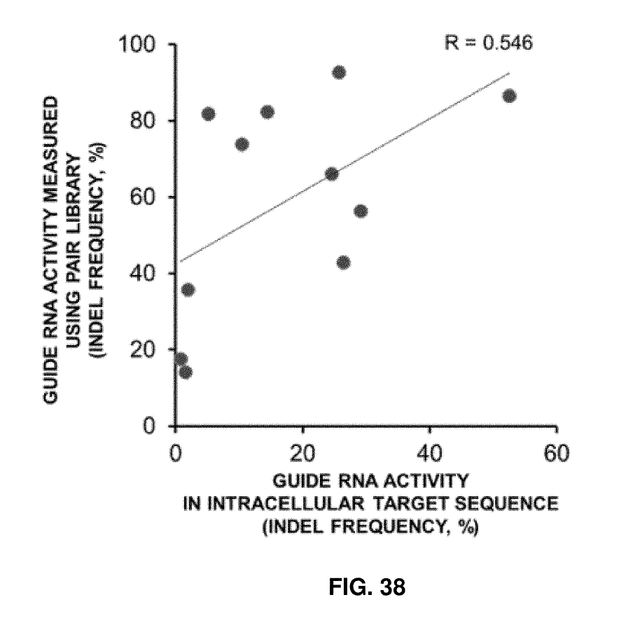

Additionally, the correlation between the degree of activity of the guide RNA obtained using the pair library of the present invention and that of the guide RNA obtained by direct analysis of the target sequences in cells was examined, and as a result, it was confirmed that they exhibited high Spearman correlation coefficients, and therefore, it was confirmed that the method of evaluating the activity of RNA-guided nucleases using the guide RNA-target sequence pair library of the present invention has high accuracy (FIG. 38).

The characteristics of the RNA-guided nucleases analyzed in present invention may include, for example,
(i) a PAM sequence of an RNA-guided nuclease,
(ii) on-target activity of an RNA-guided nuclease, or
(iii) off-target activity of an RNA-guided nuclease.

The characteristics of the RNA-guided nucleases to be analyzed may vary depending on the design of oligonucleotides, and this eventually appears as the results interpreted from the indel frequency being obtained by deep sequencing of the cell library.

In an embodiment, in a case where the PAM sequence of an RNA-guided nuclease is to be confirmed, it is possible to design oligonucleotides such that they have various nucleotide sequences and/or potential PAM sequences where the number of nucleotides of PAM sequences are different at the 5′ terminus of a target sequence during the process of these oligonucleotides. Accordingly, the PAM sequence of the corresponding RNA-guided nuclease can be confirmed by analyzing the indel frequency according to PAM sequences.

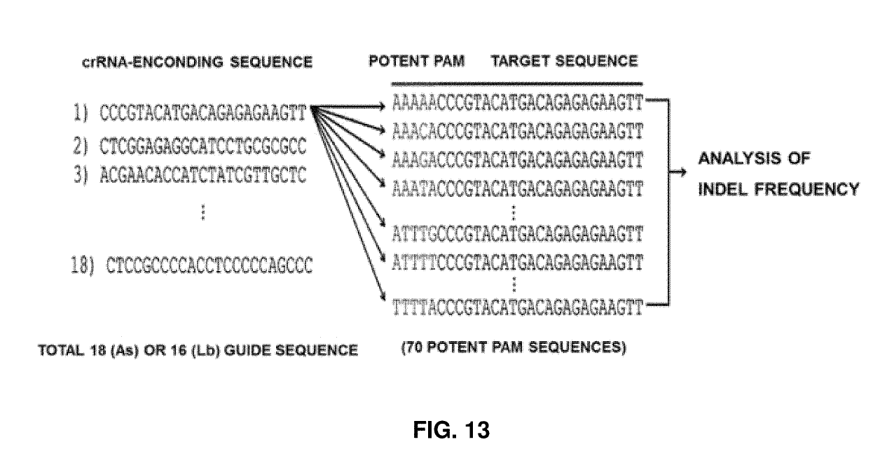

In a specific embodiment of the present invention, the PAM sequences of the Cpf1 (AsCpf1 and LbCpf1, respectively) derived from Acidaminococcus and Lachnospiraceae were analyzed using the guide RNA-target sequence pair library, and as a result, it was confirmed that TTTV, and additionally CTTA are true PAM sequences of AsCpf1 and LbCpf1 (FIGS. 13 to 19), contrary to what is previously known with regard to TTTN.

In another embodiment of the present invention, it is possible to perform analysis for the analysis of characteristics of on-target activity by designing various kinds of guide RNAs and target sequences corresponding thereto, or by varying the conditions for applying the RNA-guided nucleases. From the above, it is possible to obtain information that can maximize the target effect during the design of guide RNAs.

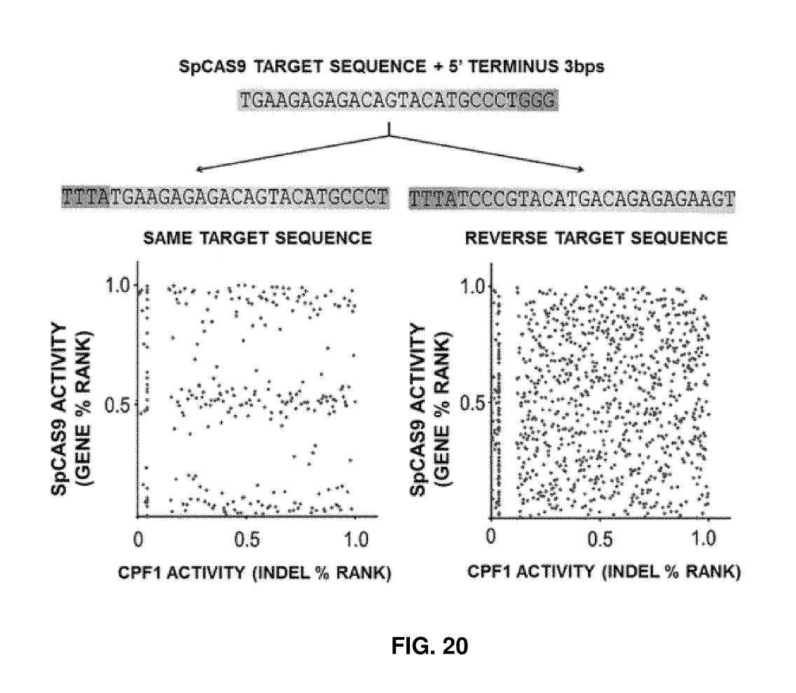

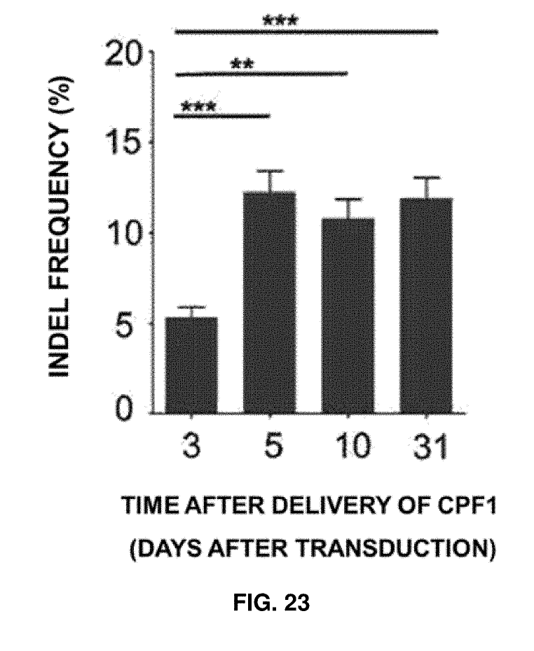

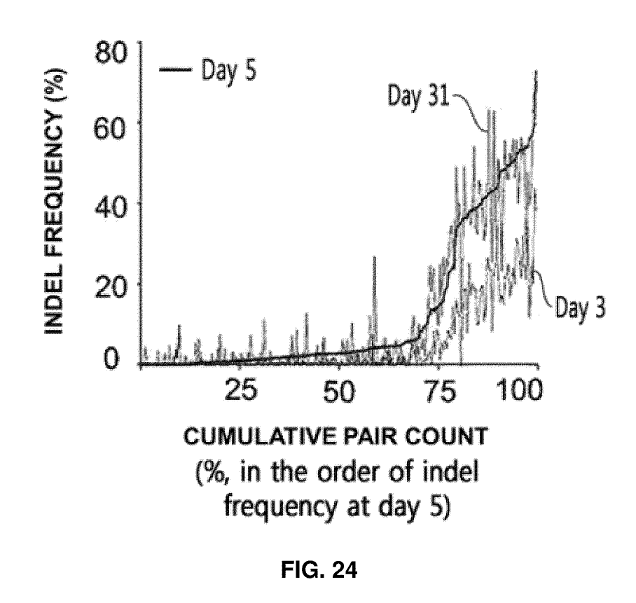

In a specific embodiment of the present invention, the characteristics of on-target activity were analyzed by varying the kinds of the RNA-guided nucleases, analyzing the positional characteristics of guide RNAs with high activity, or analyzing the GC content of a target sequence (FIGS. 20 to 22), and in another specific embodiment of the present invention, on-target activity was analyzed by varying the delivery time of lentivirus (FIGS. 23 and 24).

In another embodiment of the present invention, for the analysis of off-target activity, it is possible to design oligonucleotides such that there is a mismatch in part of the sequences between a guide RNA sequence and a target sequence, and in particular, it is possible to design by specifically differentiating the position of the target sequence. Through the above, it is possible to confirm the effect of a nucleotide mismatch according to the position of a target sequence, and this enables obtaining of information that can minimize the off-target activity during the design of a guide RNA.

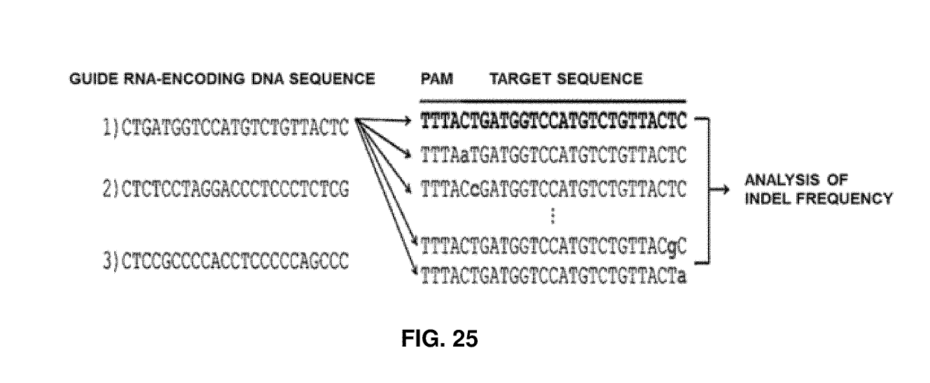

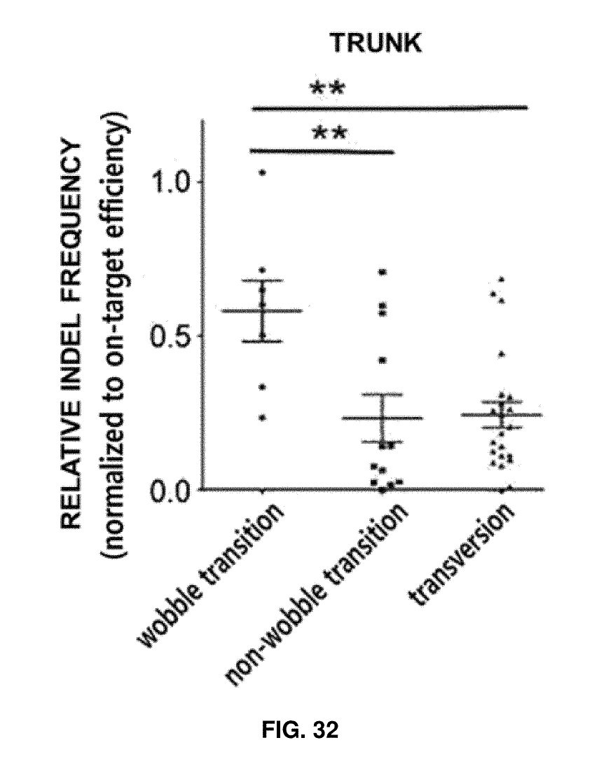

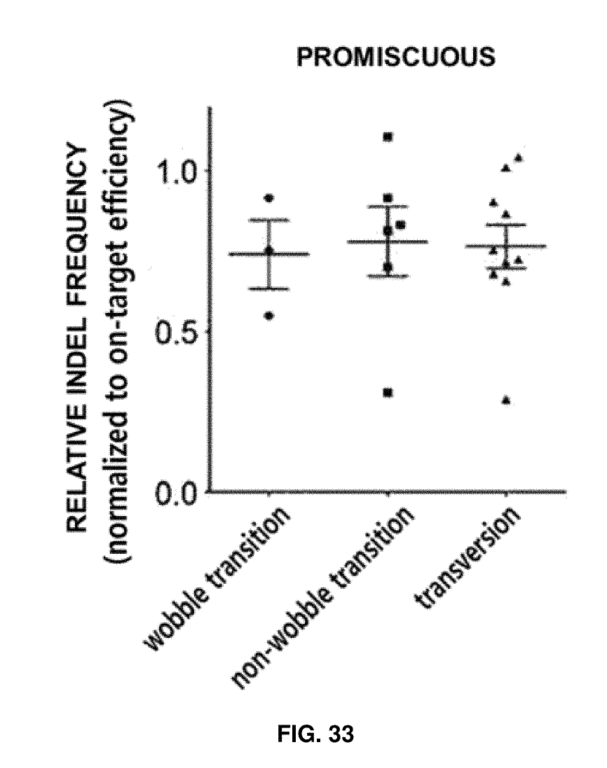

In a specific embodiment of the present invention, oligonucleotides were designed such that there is a nucleotide mismatch in guide RNA that correspond according to the position of a target sequence, and thereby the relationship between the nucleotide mismatch and off-target effects at each position of the target sequence were analyzed (FIGS. 25 to 33).

The characteristics of the RNA-guided nucleases are to provide one exemplary embodiment for evaluating the activity of RNA-guided nucleases using the guide RNA-target sequence pair library of the present invention, and the scope of the present invention should not be interpreted as being limited to the exemplary embodiments above. The characteristics of the core technology of the present invention lies in the evaluation of the activity of RNA-guided nucleases in vivo using a cell library including guide RNA-target sequence pairs in a high-throughput manner, and for this purpose, the design methods of the basic oligonucleotides and interpretations of the results thereof can be sufficiently expanded according to the intentions and purposes of those skilled in the art, the kinds of RNA-guided nucleases, etc.

Another aspect of the present invention provides a cell library including at least two kinds of cells, in which each cell includes an oligonucleotide including a guide RNA-encoding nucleotide sequence, and a target nucleotide sequence which the guide RNA targets.

Still another aspect of the present invention provides a vector including an isolated oligonucleotide, which includes a guide RNA-encoding nucleotide sequence and a target nucleotide sequence which the guide RNA targets; and a vector library.

Still another aspect of the present invention provides an oligonucleotide including a guide RNA-encoding nucleotide sequence and a target nucleotide sequence which the guide RNA targets; and an oligonucleotide library.

The cell library, vector, vector library, oligonucleotide, and oligonucleotide library are the same as described above.

Still another aspect of the present invention provides a method for constructing the oligonucleotide library, which includes: (a) setting a target nucleotide sequence, which is to be targeted with an RNA-guided nuclease; (b) designing a guide RNA-encoding nucleotide sequence, which forms a base pair with a complementary strand of the set target nucleotide sequence; (c) designing an oligonucleotide, which includes the target nucleotide sequence and a guide RNA that targets the same; and (d) repeating steps (a) to (c) at least once, and specifically two times.

The process of designing oligonucleotides for the constructing of an oligonucleotide library is the same as described above.

The process may be one in which, after determining a target sequence, the sequence of a guide RNA for the target sequence is designed, or a target sequence including a PAM sequence with regard to one guide RNA sequence is designed. That is, it is possible to analyze both on-target activity and off-target activity in the present invention, all or part of the guide RNA sequence may be perfectly complementary to the target sequence, or may be complementary to the target sequence in a state where part of the sequence is mismatched. The design process thereof may be one where several target sequences, which have a deviation in the nucleotide sequence with regard to one guide RNA in terms of the length of the sequence and/or the nucleotide sequence, are designed, and the process may be several guide RNAs, which have a deviation in the nucleotide sequence with regard to one target sequence in terms of the length of the sequence and/or the nucleotide sequence, are designed, and the process may be one such that two processes are achieved in a combined manner.

The step (c) or step (d) may include a step of synthesizing an additionally-designed oligonucleotide.

Still another aspect of the present invention provides an isolated guide RNA, which includes a sequence that is able to form a base pair with a complementary strand of a target nucleotide sequence that is adjacent to a proto-spacer-adjacent motif (PAM) sequence, that is, TTTV or CTTA.

Still another aspect of the present invention provides a composition for genome editing, which includes the isolated guide RNA, or a nucleic acid encoding the same.

The isolated guide RNA may be one where the RNA-guided nuclease used in combination is a Cpf1 protein.

Still another aspect of the present invention provides a system for genome editing in a mammalian cell, which includes the isolated guide RNA, or a nucleic acid encoding the same; and a Cpf1 protein or a nucleic acid encoding the same.

Still another aspect of the present invention provides a method for genome editing with Cpf1 in a mammalian cell, which includes sequentially or simultaneously introducing the guide RNA or a nucleic acid encoding the same; and a Cpf1 protein or a nucleic acid encoding the same, into an isolated mammalian cell.

As described above, it was confirmed in the present invention that the PAM sequences of a Cpf1 protein are TTTV or CTTA, contrary to the previous notion that it is TTTN, and thus, based on the confirmation of the present invention, the guide RNA having TTTV or CTTA as a PAM sequence can be effectively used for genome editing.

### MODE FOR THE INVENTION

Hereinafter, the present invention will be described in more detail with reference to the following Examples. However, these Examples are for illustrative purposes only and the scope of the present invention is not limited to these Examples.

### Example 1: Preparation of Pair Library for Evaluating Activity of Cpf1 and Evaluation Method Thereof

### Example 1-1: Design of Oligonucleotides

To construct a plasmid library for the evaluation of the activity of Cpf1 with regard to various guide RNAs in a high-throughput manner, 8,327 oligonucleotides for Cpf1 (AsCpf1) derived from Acidaminococcus and 3,634 oligonucleotides for Cpf1 (LbCpf1) derived from Lachnospiraceae were synthesized by the CustomArray (Bothell, Wash.). The oligonucleotides were designed such that they include a guide RNA-encoding sequence (guide sequence) and a target sequence in a length of a total of 122 to 130 nucleotides (FIG. 1).

To compare the indel frequencies at the endogenous position and the introduced position, 82 error-free oligonucleotides including an RNA-encoding sequence and a target sequence were synthesized by the Cellemics, Inc. (Seoul, Korea).

Additionally, a sequence of 27 nucleotides (SEQ ID NO: 1) and a sequence of 22 nucleotides (SEQ ID NO: 2) were included at both ends of the above oligonucleotides, respectively, so that they were able to be used as binding sites for forward and reverse primers during PCR amplification. Additionally, a unique barcode sequence with 15 nucleotides was inserted into the center of each oligonucleotide to enable recognition of each oligonucleotide. The barcode sequence was designed such that it does not include a repetition of two or more nucleotides (i.e., AA, CC, TT, and GG), and all of the barcode sequences were designed such that there is a deviation of at least two nucleotides between the barcode sequences. In each oligonucleotide, the guide RNA sequence and the target sequence were positioned upstream and downstream of the barcode sequence, respectively.

### Example 1-2: Vector Cloning

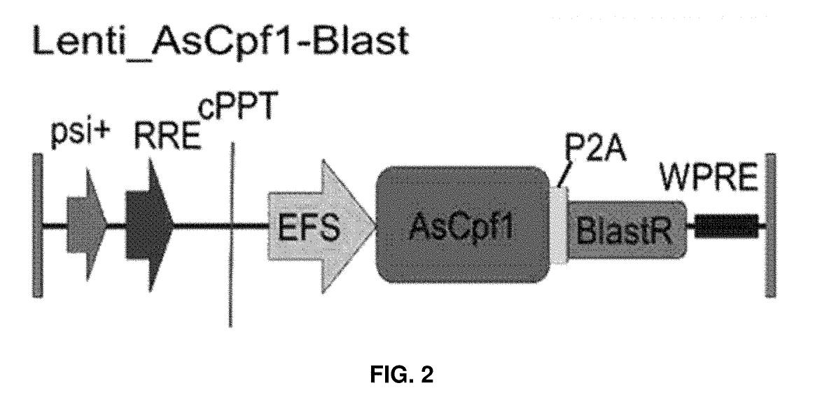

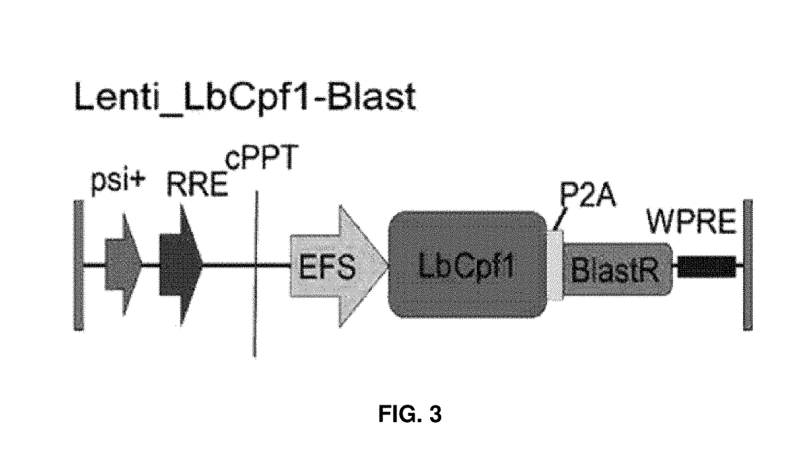

To prepare a Cpf1-expressing lentivirus vector, sequences encoding AsCpf1 and LbCpf1 derived from the plasmid (Addgene; #69982, #69988) were replicated into the lentiCas9-Blast plasmid (Addgene; #52962) and they were named as Lenti_AsCpf1-Blast (SEQ ID NO: 3) and Lenti_LbCpf1-Blast (SEQ ID NO: 4), respectively (FIGS. 2 and 3).

Additionally, to obtain a backbone vector for the preparation of a plasmid library, the SpCas9 scaffold region was removed from the lentiGuide-Puro vector (Addgene; #52963), and this vector was named as Lenti-gRNA-Puro vector (SEQ ID NO: 5) (FIG. 4).

### Example 1-3: Preparation of Plasmid Library

To prepare a plasmid library, the oligonucleotides synthesized in Examples 1 (122 and 130 nucleotides, respectively) were amplified by PCR using the Phusion polymerase (NEB) and gel purification process was performed using the MEGAquick-Spin™ Total Fragment DNA Purification Kit (Intron). Then, the Lenti-gRNA-Puro vector and a purified PCR product were assembled using the NEBuidler HiFi DNA Assembly Kit (NEB). After the assembly, the electrocompetent cells (25 μL, Lucigen) were transformed by electroporation using the above reactant (2 μL) using the MicroPulser (BioRad). Then, the transformed cells were inoculated into LB agar medium containing ampicillin (100 μg/mL), and finally, colonies corresponding to a 30-fold number of that of a library were obtained. The colonies were collected and plasmid DNA was extracted therefrom using the Plasmid Maxiprep kit (Qiagen).

### Example 1-4: Production of Lentivirus

HEK293T cells (ATCC) were cultured in 100 mm dishes coated with 0.01% poly-L-lysine (Sigma) to a level of 80% to 90% confluency. The transfer plasmid prepared in Example 3 was mixed with psPAX2 and pMD2.G in a weight ratio of 4:3:1. Then, a plasmid mixture (18 μg) was introduced into cells in 100 mm dishes using the iN-fect infection reagent (Intron Biotechnology) according to the manufacturer's directions. 15 Hours after the transfection, the medium was replaced with growth medium (12 mL). The supernatant containing the virus was collected after 39 (=15+24) and 63 (=15+48) hours from the transfection. The primary and secondary batches of the virus-containing medium were mixed, and centrifuged at 4° C. at 3,000 rpm for 5 minutes. Then, the supernatant was filtered using the Millex-HV 0.45 μm low protein binding membrane (Millipore) and stored at −80° C. until use.

### Example 1-5: Preparation of Cell Library

To prepare a cell library, lentivirus vector was transfected to HEK293T cells (1.5×10⁶ to 2.0×10⁶) which were attached to 100 mm dishes. Three days after the transduction, the cells were treated with puromycin (2 μg/mL) for 3 to 5 days. For the preservation of the library during the progress of the study, the cells containing the library were maintained at a minimum density of 3×10⁶ cells per 100 mm dish. The copy number of lentivirus vector regulatory element (WPRE) was compared with that of endogenous human gene, ALB, and the multiplicity of infection (MOI) was confirmed. To measure the copy number of provirus and ALB in a genomic DNA sample, real-time qPCR was performed using primers specific to SYBR Advantage qPCR Premix (Clontech), and WPRE or ALB. The results are shown in standard curves with lentiGuide-Puro (Addgene; #52963) and pAlbumin. To prevent the quantification bias by the plasmid DNA formation, all of the templates were digested with Ahdl before performing PCR. Since the standard plasmid DNA was used in the qPCR analysis, salmon sperm DNA was contained as the background to remedy the efficiency deviation in the quantification of genomic DNA and plasmid DNA. Although the HEK293 cells have almost 3-ploid chromosomes, the chromosome number 4 where the ALB gene is located has two pairs and thus the ratio of provirus to the cellular DNA (MOI) was calculated by copy number of WPRE/copy number of ALB×0.5.

### Example 1-6: Transduction of Cpf1 to Cell Library

For the transduction of AsCpf1- or LbCpf1-expressing lentivirus vector, first, a cell library (2×10⁶ to 3×10⁶ cells) was inoculated into 100 mm culture dishes 24 hours before transduction. Then, the AsCpf1-expressing virus vector was transduced into cells in DMEM containing 10% fetal bovine serum (FBS, Gibco), and maintained in DMEM containing 10% FBS and blasticidin S (10 μg/mL, InvivoGen).

In the case of transduction of AsCpf1- or LbCpf1-encoding plasmid, first, the cell library (3×10⁶ cells) were inoculated into three 60 mm dishes 6 hours before transduction. Then, the cells were transduced with Lenti_AsCpf1-Blast or Lenti_LbCpf1-Blast plasmid (4 μg) and Lipofectamine® 2000 (Invitrogen) (8 μL). The cells were incubated overnight and the medium was replaced with DMEM containing 10% FBS. Then, the transduced cells were cultured in culture medium containing blasticidin (10 μg/mL) from the first day of the transduction for 4 days.

### Example 1-7: Deep Sequencing

Genomic DNA was isolated from a cell library using the Wizard Genomic DNA purification kit (Promega). Then, for the analysis of indel frequency, the inserted target sequence was first amplified by PCR using the Phusion polymerase (NEB). To achieve a 100-fold or more of coverage of the cell library, the genomic DNA was used as a template in an amount of 13 μg/sample in the primary PCR (assuming that the genomic DNA for 293T cells (1×10⁶) as 10 μg). For each sample, 13 independent reactions (50 μL) were performed using the genomic DNA (1 μg) per reaction, and the reaction products were combined.

To compare the indel frequency at the endogenous site and the introduced site, 100 ng of DNA per sample was used as the DNA for the introduced target sequence and the endogenous target sequence for PCR amplification.

Then, the PCR products were purified using the MEGAquick-Spin™ Total Fragment DNA Purification Kit (Intron). In the secondary PCR, the purified product of the primary PCR (20 ng) was attached along with the Illumina adaptor and a barcode sequence. The primers used in PCR reactions are shown in Table 1 below. The final products were separated, purified, and mixed, and subjected to analysis using the MiSeq or HiSeq (Illumina).

TABLE 1

| Primer | | Sequence (5′-3′) |
|---|---|---|
| Lenti_gRNA_Puro | FP1 | CAC CGG AGA CGT TGA CTA TCG TCT CGC |
| cloning | | TAC TCT ACC ACT TGT ACT TCA GCG GTC A |
| | | (SEQ ID NO: 6) |
| | RP1 | AAG CTG ACC GCT GAA GTA CAA GTG GTA |
| | | GAG TAG CGA GAC GAT AGT CAA CGT CTC C |
| | | (SEQ ID NO: 7) |
| | FP2 | GCT TAC TCG ACT TAA CGT GCA CGT GAC |
| | | ACG TTC TAG ACC GTA CAT GCT TAC ATG |
| | | GGA TGA (SEQ ID NO: 8) |
| | RP2 | AGC TTC ATC CCA TGT AAG CAT GTA CGG |
| | | TCT AGA ACG TGT CAC GTG CAC GTT AAG |
| | | TCG AGT (SEQ ID NO: 9) |
| AsCpf1 oligo | FP | ATT TCT TGG CTT TAT ATA TCT TGT GGA AAG |
| library amplification | | GAC GAA ACA CCG TAA TTT CTA CTC TTG |
| | | TAG (SEQ ID NO: 10) |
| LbCpf1 oligo | FP | TTT CTT GGC TTT ATA TAT CTT GTG GAA AGG |
| library amplification | | ACG AAA CAC CGT AAT TTC TAC TAA GTG |
| | | TAG (SEQ ID NO: 11) |
| As/LbCpf1 oligo | RP | GAG TAA GCT GAC CGC TGA AGT ACA AGT |
| library amplification | | GGT AGA GTA GAG ATC TAG TTA CGC CAA |
| | | GCT (SEQ ID NO: 12) |
| Targeted deep | FP | ACA CTC TTT CCC TAC ACG CTC TTC |
| sequencing | | CGA TCT CTT GTG GAA AGG ACG AAA CAC C |
| | | (SEQ ID NO: 13) |
| | RP | GTG ACT GGA GTT CAG ACG TGT GCT CTT |
| | | CCG ATC TTT GTG GAT GAA TAC TGC CAT |
| | | TTG TC (SEQ ID NO: 14) |
| Indexing of Illumina | FP | AAT GAT ACG GCG ACC GAG ATC TAC |
| | | AC (SEQ ID NO: 15) - (8 bp barcode sequence) - ACA |
| | | CTC TTT CCC TAC ACG AC (SEQ ID NO: 16) |
| | RP | CAA GCA GAA GAC GGC ATA CGA GAT |
| | | (SEQ ID NO: 17) - (8 bp barcode) - GTG ACT GGA GTT |
| | | CAG ACG TGT (SEQ ID NO: 18) |
| qPCR for WPRE | FP | GAT ACG CTG CTT TAA TGC CTT TG (SEQ ID NO: 19) |
| | RP | GAG ACA GCA ACC AGG ATT TAT ACA AG (SEQ ID |
| | | NO: 20) |
| qPCR for ALB | FP | GCT GTC ATC TCT TGT GGG CTG T (SEQ ID NO: 21) |
| | RP | ACT CAT GGG AGC TGC TGG TTC (SEQ ID NO: 22) |
| Endogenous target | FP | TTG CTG TGG CAG AGC CAG CG (SEQ ID NO: 29) |
| 1-5 | RP | TTG CTT CAC TTT AAT CCT TTC TTG CAG (SEQ ID |
| | | NO: 30) |
| Endogenous target | FP | CTC CTG CAA GAA AGG ATT AAA |
| 6~10 | | GTG (SEQ ID NO: 31) |
| | RP | ACC TAC CTA ATA GTT ACT TCC TGA AGG |
| | | G (SEQ ID NO: 32) |
| Endogenous target | FP | CTC GTT CTT TCC ATC AAA TAG TGT GGT |
| 11~14 | | G (SEQ ID NO: 33) |
| | RP | CTG CAG TAA TTG TTA CTC TGT GTC TTC |
| | | C (SEQ ID NO: 34) |
| Endogenous target | FP | TTG AGC TGA CCC ATA AAT ACA |
| 15~17 | | GG (SEQ ID NO: 35) |
| | RP | CCC TCT TAA CTG GAT CAG CAA |
| | | CGG (SEQ ID NO: 36) |
| Endogenous target | FP | TGG GGT CGC CAT TGT AGT TCC C (SEQ ID NO: 37) |
| 18 | RP | GTC ACA AAG ATC AGC ATC AGG CAT |
| | | GG (SEQ ID NO: 38) |
| Endogenous target | FP | CGT TCA CCT GGG AGG GGA AG (SEQ ID NO: 39) |
| 19~22 | RP | TCT GCA AAG AAC TTT ATT CCG AGT AAG |
| | | C (SEQ ID NO: 40) |
| Endogenous target | FP | CCC AAA AGA CAT ATT CAC CCA GAA TCC |
| 23~28 | | C (SEQ ID NO: 41) |
| | | RP CAA CAT CAA GGT GTG GGC AGG GCT |
| | | GC (SEQ ID NO: 42) |
| Endogenous target | FP | ACC TGG AGT CTG CAG AGC TGG (SEQ ID NO: 43) |
| 29~30 | RP | AAG CGG TAA ACA AAG GAT AGC |
| | | TGG (SEQ ID NO: 44) |
| Endogenous target | FP | CCA TGG GAA ACG AAT ACA GGT CTC G (SEQ ID |
| 31~35 | | NO: 45) |
| | RP | CTT CAG AAG AAA AAC CTC CAC TC (SEQ ID NO: |
| | | 46) |
| Endogenous target | FP | AAC TGA GAA ACA GCC AGA GAG GAA G (SEQ ID |
| 36~37 | | NO: 47) |
| | RP | CAT CTG ATG CTG ACT CAG AGC GC (SEQ ID NO: |
| | | 48) |
| Endogenous target | FP | GCT GCC ACC CCC TGC TC (SEQ ID NO: 49) |
| 38~ 42 | RP | ATC AGA ATG AAA AAT CTC ACC CCT CC (SEQ ID |
| | | NO: 50) |
| Endogenous target | FP | GTC TCC GTG ATG GGG GTG G (SEQ ID NO: 51) |
| 43~46 | RP | CTG CCT TGT AAG ACT TTA AAT ATT CTG |
| | | CTC C (SEQ ID NO: 52) |
| Endogenous target | FP | AAG CCA TAT TCA GTT TTA GGG AAA |
| 47~48 | | AGC (SEQ ID NO: 53) |
| | RP | ATT TCC AAG TAA GCT GCA AGG AAA |
| | | GC (SEQ ID NO: 54) |
| Endogenous target | FP | AAG TCT TAC AAG GCA GAG TAA AGA |
| 49~52 | | TC (SEQ ID NO: 55) |
| | RP | GCA GGG TAA AAC AAT CGG ACC (SEQ ID NO: 56) |
| Endogenous target | FP | CAA CCA CCT CAG AAG AGC CAG ATT |
| 53~57 | | CC (SEQ ID NO: 57) |
| | RP | CTC TGT AGT TAT TTG AGC AAT GCC |
| | | AC (SEQ ID NO: 58) |
| Endogenous target | FP | CAG TGA ATA TAC AGG ATT GGG GTT |
| 58~64 | | GTG (SEQ ID NO: 59) |
| | RP | ACA ACT GGT AAG GTG GGC CCA GG (SEQ ID NO: |
| | | 60) |
| Endogenous target | FP | CAA GCA CAA ACA AAT CAG GCT AAA TCC |
| 65~72 | | (SEQ ID NO: 61) |
| | RP | CCC TGA GCT TGG GGG AGA GTT AC (SEQ ID NO: |
| | | 62) |
| Endogenous target | FP | TCC TCT GGG GAA AGA GTG GCC (SEQ ID NO: 63) |
| 73~78 | RP | TGT GGG GTC GTT CCT GAT GAA AC (SEQ ID NO: |
| | | 64) |
| Endogenous target | FP | AAC TGG TTT AGC TAG TGC ATA CAT |
| 79~82 | | GC (SEQ ID NO: 65) |
| | RP | GGT GGG AGT TTC TGT TAC AGG CAA C (SEQ ID |
| | | NO: 66) |

FP: forward primer, RP: reverse primer.

### Example 1-8: Analysis of Pair Copy Number

For the evaluation of copy number of each pair in a library, the readings were normalized using the following equation.

the number of normalized reading per pair=(the number of reading per pair/total number of readings for all of the pairs in a sample)×10⁶+1

### Example 1-9: Analysis of Indel Frequency

Deep sequencing data was classified and analyzed using the custom Python scripts. Data classification of each guide RNA-target pair was performed based on a 15-bp barcode sequence and a 4-bp constant sequence downstream thereof (i.e., a total 19-bp sequence). The insertion or deletion located in the periphery of the expected cleavage site (i.e., an 8 bp region in the middle of the cleavage site) was considered as a mutation induced by Cpf1. Single nucleotide substitution was removed from the analysis. The actual indel frequency derived from the activity of Cpf1 and guide RNA was calculated by deducting the background indel frequency with the cell library in which Cpf1 was not delivered in the observed indel frequency. The background indel frequency mostly occurs in the synthesis of oligonucleotides. To increase the accuracy of analysis, the deep sequencing data was classified according to the number of reading and the background indel frequency per pair (Table 2).

TABLE 2

| | Minimum | Maximum value permitting |
|---|---|---|
| | reading | background indel frequency |
| Purpose | per pair | to be removed from analysis |
| Confirmation of AsCpf1 PAM | 100 | 8% |
| Confirmation of LbCpf1 PAM | 30 | 8% |
| Profiling of on-target effect of | 100 | 8% |
| AsCpf1 | | |
| Profiling of off-target effect of | 100 | 8% |
| AsCpf1 | | |
| Analysis of time-dependent | 300 | 8% |
| indel frequency | | |
| Profiling of off-target effect of | 300 | 8% |
| guide RNA fragment | | |

### Example 1-10: Comparison of Indel Frequency

HEK293T cells were seeded into a 48-well dish and transduced with an independent lentivirus vector containing a guide RNA-encoding sequence and a target sequence. After 3 days of the transduction, the cells were treated with puromycin (2 pg/mL) to remove the cells which were not transduced. Cpf1 was delivered to the transduced cells using the AsCpf1-expressing lentivirus vector as described above. Five days after the Cpf1 introduction, DNA was isolated from the cells and was subjected to deep sequencing.

### Example 1-11: Calculation of Chromatin Accessibility

Except the chromosome nos. 17 and 22, where 4 copies are present per cell, 4 genome regions were randomly selected. A total of 82 guide RNAs were designed such that they target random loci within the four regions. The DNase I sensitivity score was calculated using the DNase-seq (ENCFF000SPE) data drawn from Encyclopedia of DNA element (ENCODE). The DNase I sensitivity score at each position of the target region was calculated by first counting the overlapping the number of DNase-seq sequencing read fragments at the corresponding position.

For example, when there are two sequencing reading overlaps at the position 5 of the target region, the score at the above position was assumed to be 2. Each region including the PAM and target sequences has a length of 27 bp. As such, the DNase I sensitivity score at the target region was obtained by averaging the 27 scores at each position.

When the DNase I scores at the 82 target regions within 3.2 billion positions of human's genome (hg19/GRCh37 from UCSC genome browser), the scores were shown to be widely distributed (0% to 99.99%).

### Example 2: Preparation of Pair Library for Evaluating Activity of Cas9 and Evaluation Method Thereof

The present inventors have confirmed the method of evaluating activity of the RNA-guided nucleases of the present invention using SpCas9, which is a different kind of RNA-guided nuclease.

### Example 2-1: Design of Oligonucleotides

To construct a plasmid library for the evaluation of SpCas9 activity with regard to various guide RNAs in a high-throughput manner, the present inventors have designed guide RNA-target sequence oligonucleotides by a method similar to Examples described above.

Specifically, 89,592 oligonucleotides were synthesized for the Cas9 derived from Streptococcus pyogenes (SpCas9) by CustomArray (Bothell, Wash.) and Twist Bioscience (San Francisco, Calif.). The oligonucleotides had a total length of 120 nucleotides and they were designed to include a guide RNA-encoding sequence (guide sequence) and a target sequence (FIG. 35). Additionally, a sequence of 26 nucleotides (TATCTTGTGGAAAGGACGAAACACCG, SEQ ID NO: 23) and a sequence of 29 nucleotides (GTTTTAGAGCTAGAAATAGCAAGTTAAAA, SEQ ID NO: 24) were included at both ends of the above oligonucleotides, respectively, so that they were able to be used as binding sites for forward and reverse primers during PCR amplification. Additionally, a unique 15-bp barcode sequence was inserted into the center of each oligonucleotide for the identification of each oligonucleotide. The barcode sequence was designed such that it does not include a repetition of two or more nucleotides (i.e., AA, CC, TT, and GG), and all of the barcode sequences were designed such that there is a deviation of at least two nucleotides between the barcode sequences. In each oligonucleotide, the target sequence and the guide RNA were positioned upstream and downstream of barcode sequence, respectively.

### Example 2-2: Preparation of Plasmid Library

To prepare a plasmid library including the oligonucleotides prepared in Examples above, the oligonucleotides (each of 120 nucleotides) were amplified by PCR using the Phusion polymerase (NEB), and gel purification process was performed using the MEGAquick-Spin™ Total Fragment DNA Purification Kit (Intron). Then, the LentiGuide_Puro (Addgene, #52963) vector and the purified PCR products were assembled using the NEBuidler HiFi DNA Assembly Kit (NEB). After the assembly, the electrocompetent cells (2 μL, Lucigen) were transformed by electroporation using the above reactant (2 μL) using the MicroPulser (BioRad). Then, the transformed cells were inoculated into LB agar medium containing ampicillin (100 μg/mL), and finally, colonies corresponding to a 17 to 18-fold number of that of a library were obtained. The colonies were collected and plasmid DNA was extracted therefrom using the Plasmid Maxiprep kit (Qiagen).

### Example 2-3: Production of Lentivirus

HEK293T cells (ATCC) were cultured in 100 mm dishes coated with 0.01% poly-L-lysine (Sigma) to a level of 80% to 90% confluency. The transfer plasmid prepared in Example 2-2 was mixed with psPAX2 and pMD2.G in a weight ratio of 4:3:1. Then, a plasmid mixture (18 μg) was introduced into cells in 100 mm dishes using the iN-fect infection reagent (Intron Biotechnology) according to the manufacturer's directions. 15 Hours after the transfection, the medium was replaced with growth medium (12 mL). The supernatant containing the virus was collected after 39 (=15+24) and 63 (=15+48) hours from the transfection. The primary and secondary batches of the virus-containing medium were mixed, and centrifuged at 4° C. at 3,000 rpm for 5 minutes. Then, the supernatant was filtered using the Millex-HV 0.45 μm low protein binding membrane (Millipore) and stored at −80° C. until use.

### Example 2-4: Preparation of Cell Library

To prepare a cell library including the oligonucleotides, the lentivirus vector prepared in Examples above was transfected to HEK293T cells (7.0×10⁶ cells/dish) which were attached to three 150 mm dishes. Three days after the transduction, the cells were treated with puromycin (2 μg/mL) for 3 to 5 days. For the preservation of the library during the progress of the study, the cells containing the library were maintained at a cell density (7.0×10⁶ cells/dish) in three 150 mm dishes.

### Example 2-5: Transfer of Cas9 to Cell Library

For the transduction of SpCas9-expressing lentivirus vector, the cell library (2.1×10⁷ cells) prepared in Examples above were first inoculated into three 150 mm culture dishes 24 hours before transduction.

Then, the SpCas9-expressing virus vector was transduced into cells in DMEM containing 10% fetal bovine serum (FBS, Gibco), and maintained in DMEM containing 10% FBS and blasticidin S (10 μg/mL, InvivoGen).

### Example 2-6: Deep Sequencing

Genomic DNA was isolated from the cell library prepared in Examples above using the Wizard Genomic DNA purification kit (Promega).

Then, for the analysis of indel frequency, the inserted target sequence was first amplified by PCR using the Phusion polymerase (NEB). To achieve a 100-fold or more of coverage of the cell library, the genomic DNA was used as a template in an amount of 180 μg/sample in the primary PCR (assuming that the genomic DNA for 293T cells (1×10⁶) as 10 μg). For each sample, 90 independent reactions (50 μL) were performed using the genomic DNA (2 μg) per reaction, and the reaction products were combined.

Then, the PCR products were purified using the MEGAquick-Spin™ Total Fragment DNA Purification Kit (Intron).

In the secondary PCR, the purified product of the primary PCR (20 ng) was attached along with the Illumina adaptor and a barcode sequence. The primers used in PCR reactions are shown in Table 3 below. The final products were separated, purified, and mixed, and subjected to analysis using the MiSeq or HiSeq (Illumina).

TABLE 3

| Primer | | Sequence (5′-3′) |
|---|---|---|
| SpCas9 oligo | FP | TTG AAA GTA TTT CGA TTT CTT GGC TTT ATA |
| library amplification | | TAT CTT GTG GAA AGG ACG AAA CAC C (SEQ ID NO: |
| | | 25) |
| | RP | TTT CAA GTT GAT AAC GGA CTA GCC TTA TTT |
| | | TAA CTT GCT ATT TCT AGC TCT AAA AC (SEQ ID NO: |
| | | 26) |
| Targeted deep | FP | ACA CTC TTT CCC TAC ACG CTC TTC CGA |
| sequencing | | TCT TGG ACT ATC ATA TGC TTA CCG TAA CTT G |
| (SpCas9) | | (SEQ ID NO: 27) |
| | RP | GTG ACT GGA GTT CAG ACG TGT GCT CTT CCG |
| | | ATC TTT TGT CTC AAG ATC TAG TTA CGC CAA G |
| | | (SEQ ID NO: 28) |

The present inventors have performed the evaluation of Cas9 activity in a manner similar to that for the evaluation of Cpf1 activity in Examples above, using the pair library prepared in Examples above.

### Experimental Example 1: Evaluation of Cpf1 Activity Using Pair Library

### Experimental Example 1-1: Development of Guide RNA-Target Sequence Pair Library

For the evaluation of Cpf1 activity along with various guide RNAs in a high-throughput manner, the present inventors have prepared a guide RNA-target sequence pair library. They have amplified by PCR a pool of 11,961 array-synthesized oligonucleotides including the target sequences and the guide RNA sequence corresponding thereto (FIG. 1), and cloned with a lentivirus plasmid using the Gibson assembly (FIG. 4). The direct repeat sequence (SEQ ID NO: 20) is a position to which the forward primer binds, and the guide sequence is a sequence for crRNA. The target sequence includes a PAM sequence, and the constant sequence (SEQ ID NO: 21), being a constant region vector annealing site, is a position to which the reverse primer binds. The sequence of the plasmid cloned through the above process has the nucleotide sequence of SEQ ID NO: 3.

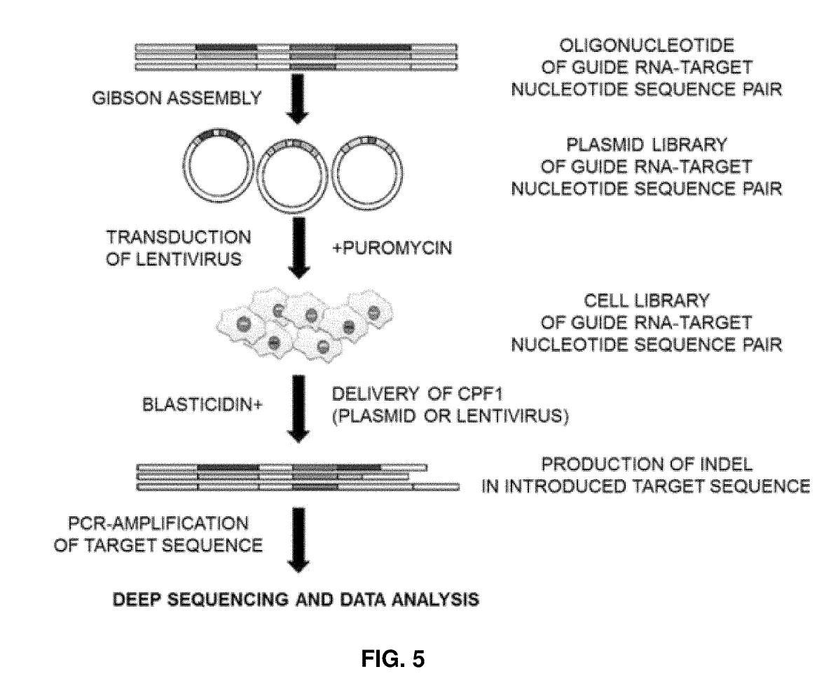

To prepare a cell library, which expresses a guide RNA and includes its corresponding sequence in the genome, the lentivirus library prepared from the plasmid library was treated on the HEK293T cells (FIG. 5). Then, to induce the cleavage by the guide RNA and indel formation to the target sequence inserted into the genome, the Cpf1-encoding plasmid was transduced into cells or the Cpf1-expressing lentivirus vector was transduced into cells thereby delivering Cpf1 to the cell library.

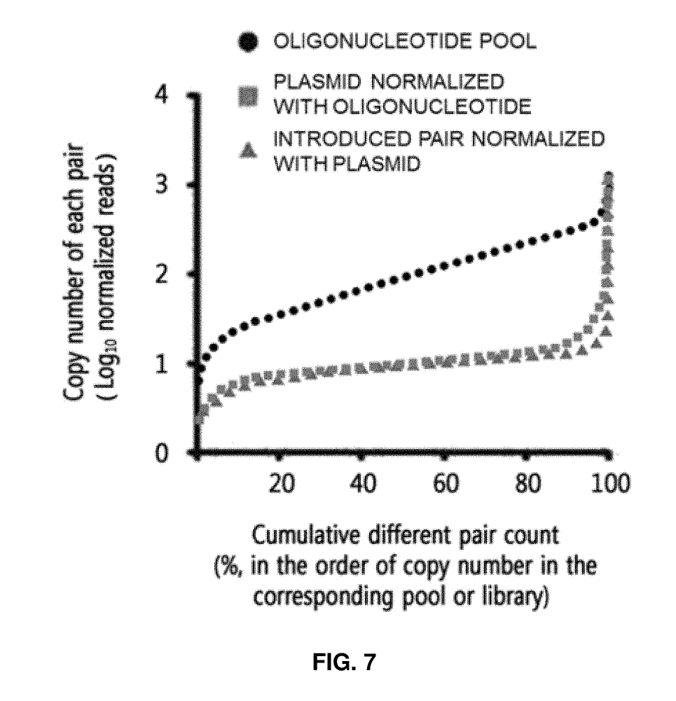

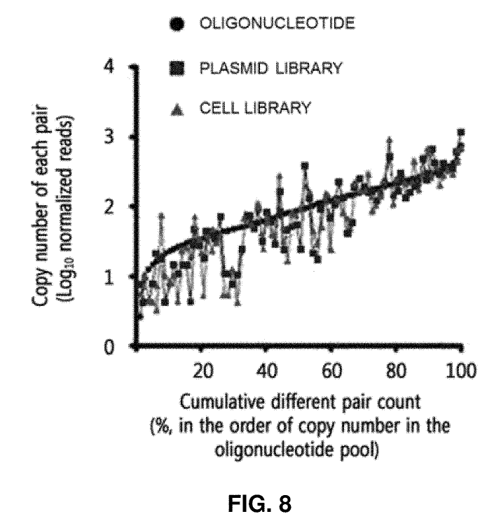

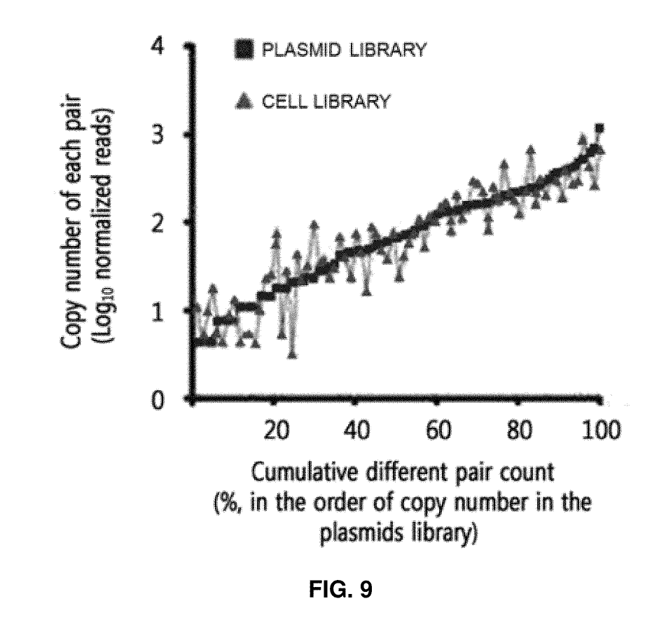

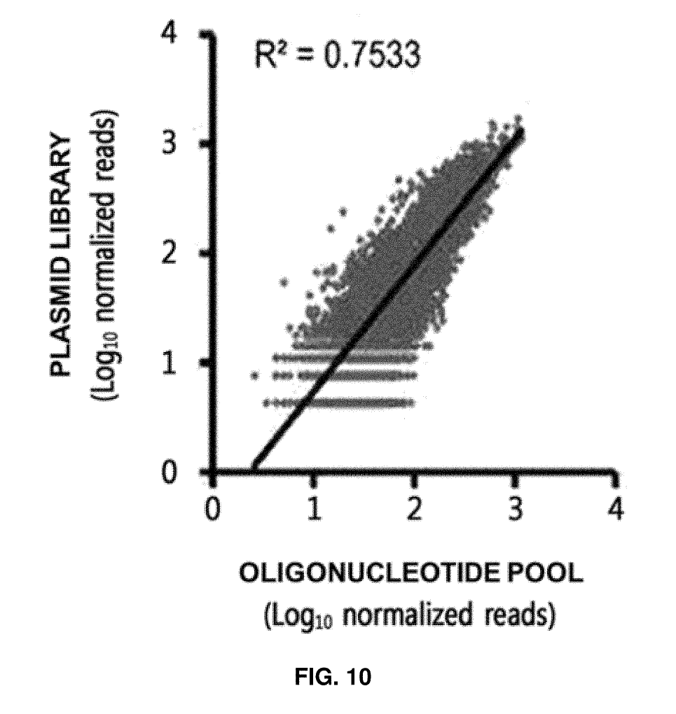

Then, the target sequence was amplified by PCR, and deep sequencing-based analysis was performed for the evaluation of indel frequency. As a result, it was confirmed through deep sequencing that the relative copy number of each pair varies in the pool of oligonucleotides. That is, based on the copy number, the copy number showed a deviation of up to the maximum of 130-fold in 99% of the oligonucleotides, excluding the top 0.5% of oligonucleotides having the highest copy number and the bottom 0.5% of oligonucleotides having the lowest copy number (FIG. 6). The plasmid and cell libraries showed a slightly higher level of deviation in copy number compared to that of the oligonucleotide pool. As such, the pair copy numbers of the plasmid and cell libraries were standardized relative to the pair copy number of the oligonucleotide and the plasmid, respectively. As a result, it was confirmed that a low level of deviation was shown compared to the deviation of copy number of the oligonucleotide pool (FIG. 7). The deviation that occurs additionally in most of the copy number during the process of forming the plasmid library and cell library was shown to be within the range of pair copy number deviation of the oligonucleotide and plasmid libraries, respectively (FIGS. 8 and 9). The copy number of each pair in the oligonucleotide pool, plasmid library, and cell library showed a very high correlation (FIGS. 10 to 12). To summarize, these deviations increase along with the progress of the processes of preparing a cell library (i.e., Gibson assembly, transformation, preparation of lentivirus vector, transduction, etc.) and the deviation in copy number of each pair in a cell library is mostly caused by the copy number deviation of the oligonucleotide. Meanwhile, the MOI in the cell library was shown to be about 7.0.

The following Table 4 provides a summary of conditions for design and filtering of oligonucleotides for the analysis purpose.

TABLE 4

| | | | | | Number of | |
|---|---|---|---|---|---|---|
| | Number | | | | different | Number of |
| | of | | Number | PAM | guide | different guide |
| Category | designed | Filtering | of filtered | sequence | sequences | sequences |
| (Purpose) | pairs | conditions | pairs | used | designed | after filtering |
| Determination | 1,540 | 100 or more | 1,074 | 70 | 22 | 18 |
| of PAM of | | (read), 8% | | different | | |
| AsCpf1 | | or less | | types | | |
| | | Background | | | | |
| | | indels | | | | |
| Determination | 1,540 | 30 or more | 940 | 70 | 22 | 16 |
| of PAM of | | (read), 8% | | different | | |
| LbCpf1 | | or less | | types | | |
| | | Background | | | | |
| | | indels | | | | |
| AsCpf1 | 2,381 | 100 or more | 1,251 | ATTTA | 2,381 | 1,251 |
| activity | | (read), 8% | | | | |
| | | or less | | | | |
| | | Background | | | | |
| | | indels | | | | |
| AsCpf1 | 420 | 300 or more | 315 | ATTTA | 7 | 7 |
| activity using | | (read), | | | | |
| truncated | | 8% or less | | | | |
| guide | | Background | | | | |
| | | indels | | | | |
| AsCpf1 | 2,580 | 100 or more | 1,543 | ATTTA | 4 | 3 |
| activity for | | (read), 8% | | | | |
| mismatched | | or less | | | | |
| target | | Background | | | | |
| sequence | | indels | | | | |
| LbCpf1 | 1,342 | 30 or more | 742 | ATTTA | 4 | Not analyzed |
| activity for | | (read), 8% | | | | due to |
| mismatched | | or less | | | | insufficient |
| target | | Background | | | | reading |
| sequence | | indels | | | | number |
| Comparison | 8,327 | 100 or more | 156 | 70 | 3,794 | 47 |
| of indel | | (read), 8% | | different | | |
| frequency in | | or less | | types | | |
| biological | | Background | | | | |
| replicate | | indels | | | | |
| Comparison of | 8,327 | 200 or more | 233 | 70 | 3,794 | 49 |
| indel | | (read), 8% | | different | | |
| frequency | | or less | | types | | |
| between two | | Background | | | | |
| different | | indels | | | | |
| methods of | | | | | | |
| Cpf1 delivery | | | | | | |

The following Table 5 shows a table in which the number of pairs in oligonucleotide pool and cell library are summarized.

TABLE 5

| | AsCpf1 | | LbCpf1 | |
|---|---|---|---|---|
| | oligonucleotide | | oligonucleotide | |
| Category | pool | cell library | pool | cell library |
| Number of designed pairs | 8,327 | | 3,634 | |
| Number of pairs included (1 | 8,313 | 8,146 | 3,626 | 3,497 |
| or more: read) | | | | |
| Percentage of | 99.8% | 97.8% | 99.8% | 96.2% |
| included/designed pairs (%) | | | | |
| Number of total reads by | 1,238,978 | 10,378,634 | 475,610 | 584,771 |
| deep sequencing | | | | |

### Experimental Example 1-2: Comparison of Indel Frequencies at Endogenous Target Position and Introduced Position

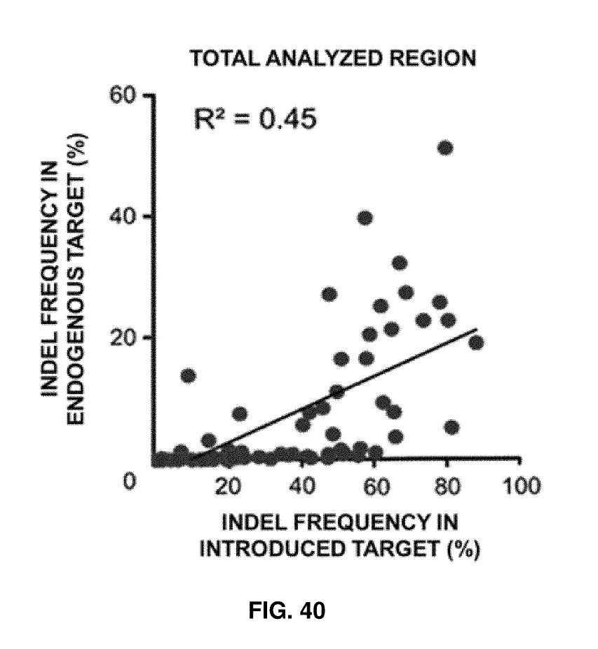

The present inventors have confirmed that there is a strong correlation between indel frequencies of a particular target sequence positioned at the endogenous genome site and the introduced synthesis site by the corresponding lentivirus (FIG. 40). Such a high correlation showed a higher level compared to when a library not forming a pair was used.

Although the chromatin accessibility that affects the efficiency of Cas9-mediated indel formation varies depending on the endogenous region, the lentivirus is integrated more in active transcription region, and thus the chromatin accessibility is expected to be higher in the introduced region. To reduce the changes in indel frequency due to the deviation of chromatin accessibility in the endogenous region, the present inventors compared the correlation between indel frequencies in a subset of the endogenous region and the introduced region with similar chromatin accessibility.

For this purpose, the chromatin accessibility of HEK293T cells was calculated using the DNase I sensitive data obtained from the DNaseOseq value obtained from the Encyclopedia of DNA element (ENCODE).

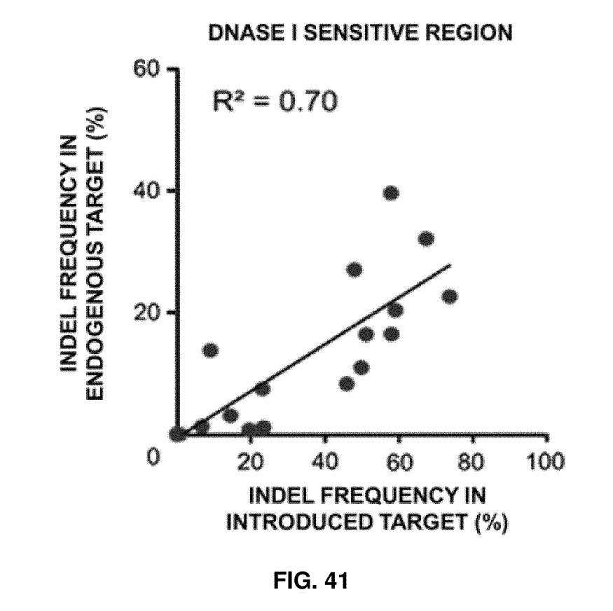

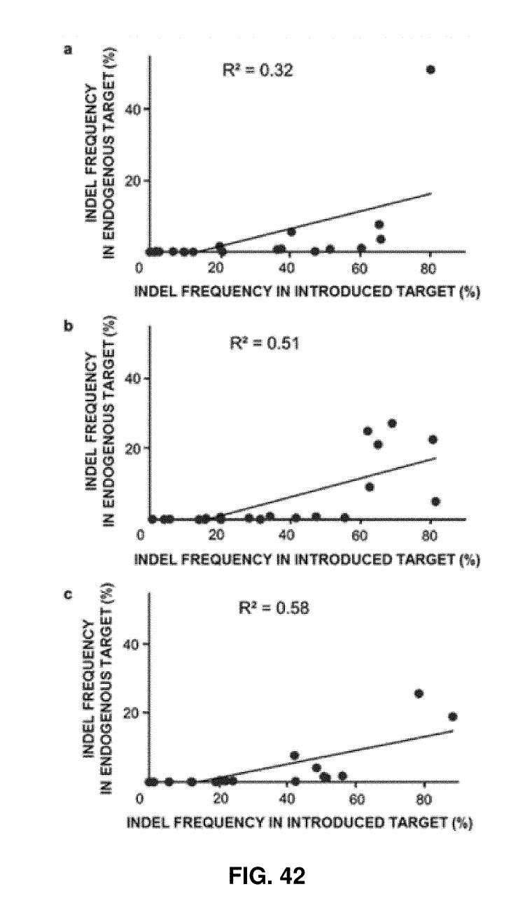

As a result, it was confirmed that the correlation was higher in the target region subset with a similar chromatin accessibility score, and in particular, it was even higher at the subset with higher chromatin accessibility (FIGS. 41 and 42). In most target sequences, the indel frequency in the introduced sequence was higher than that at the endogenous target region, and in particular, was higher in the region with low chromatin accessibility.

Additionally, with regard to the copy number of each constituting element, the cell library showed volatility similar to the libraries used in the previous studies (FIGS. 6 to 11).

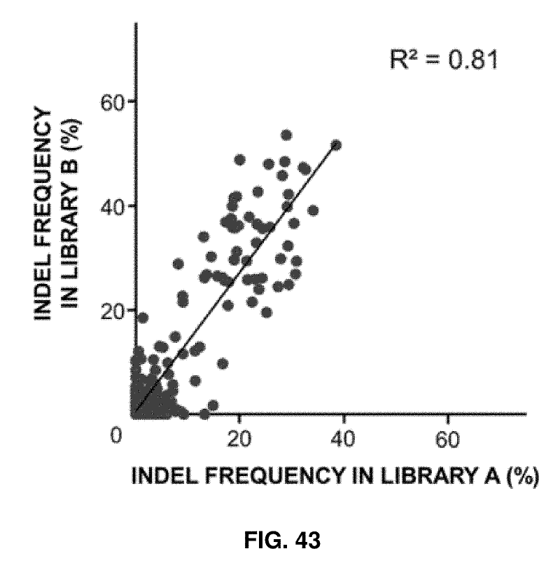

Meanwhile, the average MOI of the cell libraries was about 7.0, and there was a strong correlation between the two biological replicates. The delivery of Cpf1 with regard to the two different cell libraries caused a similar indel frequency (FIG. 43).

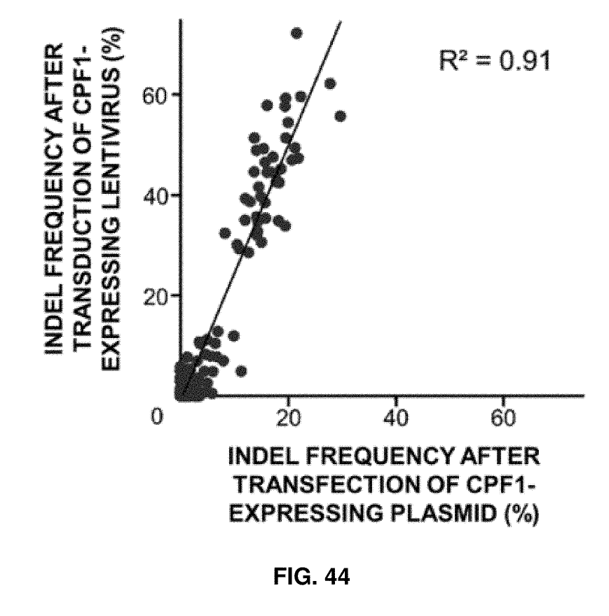

Additionally, the present inventors have confirmed that there is a clear correlation in indel frequency when Cpf1 was delivered by two different methods (i.e., transient transfection of a Cpf1-encoding plasmid and transduction of a Cpf1-encoding lentivirus vector) (FIG. 44).

In most of the analyzed target sequences, it was confirmed that the indel frequency became higher after the transduction of the Cpf1-encoding lentivirus vector (FIG. 44).

Accordingly, the present inventors have conducted experiments by means of Cpf1 transduction through the lentivirus vector, except the experiment on determining the LbCpf1 PAM which was conducted by transient plasmid transfection.

### Experimental Example 1-3: Confirmation of PAM Sequence in Mammalian Cells

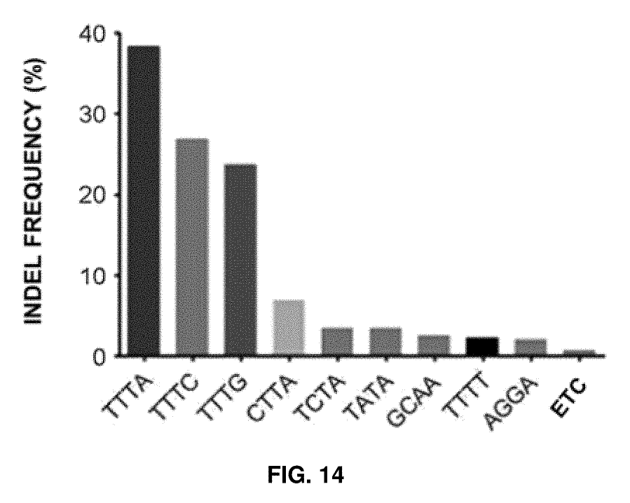

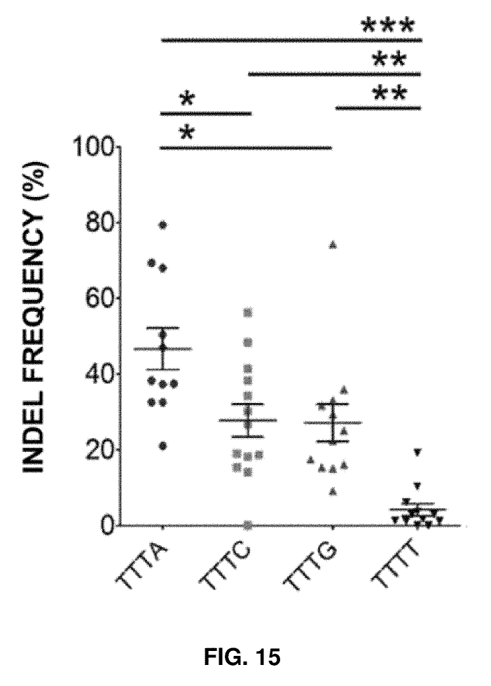

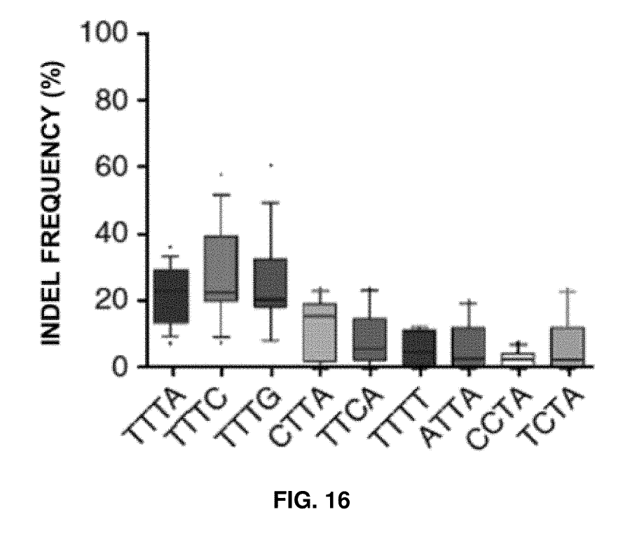

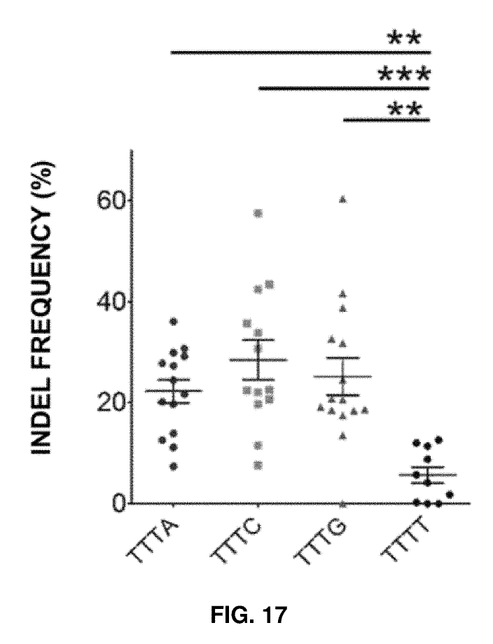

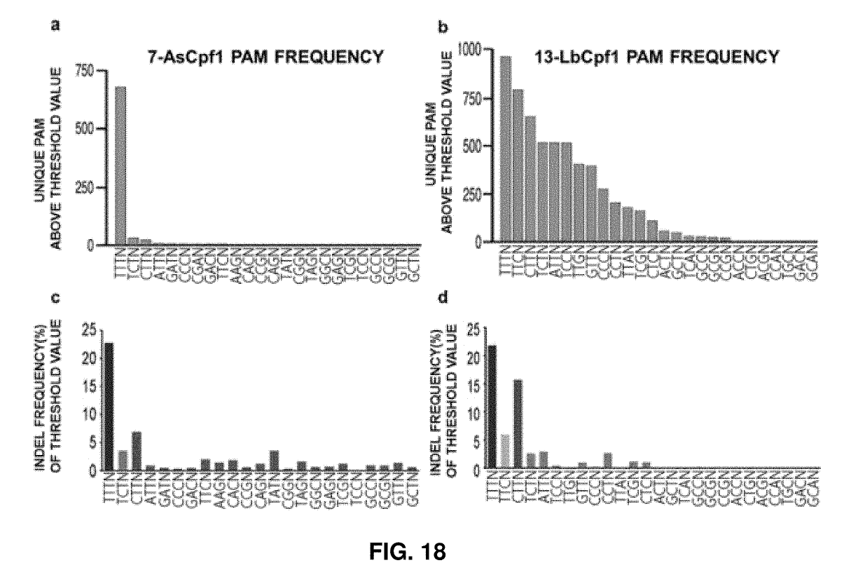

The present inventors have attempted to confirm the protospacer adjacent motif (PAM) sequence utilized by Cpf1 derived from Acidaminococcus (As) or Lachnospiraceae (Lb) by in vivo system of the present invention. Until today, the PAM sequence, which is used by RNA-programmable nucleases, has been confirmed only in in vitro conditions or in a bacterial system, not in mammalian cells. When the Cpf1 derived from As and Lb was analyzed in in vitro conditions, 70 (i.e., 4³ (indicated as ANNNA)+3 (indicated as ATTTB)+3 (indicated as BTTTA)) mutually-different PAM sequences were prepared with regard to 18 (As) or 16 (Lb) guide sequences, considering that TTTN is the most-frequently-used PAM sequence and the structure of AsCpf1 supports TTTN as a potential PAM sequence (a total of 1,260 (70×18) target sequences for AsCpf1; and a total of 1,120 (70×16) target sequences for LbCpf1, FIG. 13). As a result, the highest indel frequency was shown in both AsCpf1 (FIGS. 14 and 15) and LbCpf1 (FIGS. 16 and 17), when TTTA, TTTC, or TTTG was used as a PAM sequence, except TTTT, in HEK293T cells. These results suggest that TTTV, not TTTN, is the PAM sequence most frequently used in mammalian cells by the above two enzymes. Additionally, except TTTV, CTTA showed the highest indel frequency for Cpf1 derived from As and Lb, and can be considered as a secondary PAM sequence. The deviation in the PAM sequences used in in vitro conditions and mammalian cell conditions (FIG. 18) agreed with the deviation in the genome editing efficiency between the two systems, and it suggests that it is very important to verify the PAM sequence in a mammalian cell, not in vitro, so as to establish an efficient method for editing mammalian genome.

The co-crystal structure of AsCpf1, crRNA, and target DNA represents that the first three nucleotides (5′-TTT-3′) not including forth nucleotide of PAM sequence interacts with the Cpf1 protein, and supports the “5′-TTTN-3′” as a PAM sequence. The in vivo verification study of the present inventors helps to understand the PAM preference from TTTN to TTTV in mammalian cells.

Additionally, with regard to the indel frequency of AsCpf1 (not the indel frequency of LbCpf1), it was confirmed that when TTTA was used as a PAM sequence, there was a high significance in a low level. This suggests that TTTA has a slightly higher preference as a PAM sequence of AsCpf1 to other potential PAM sequences.

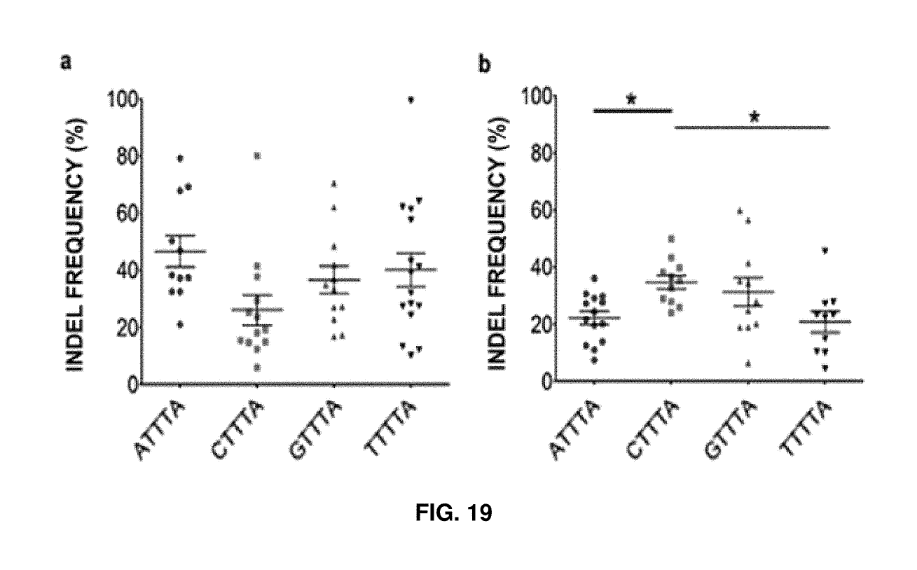

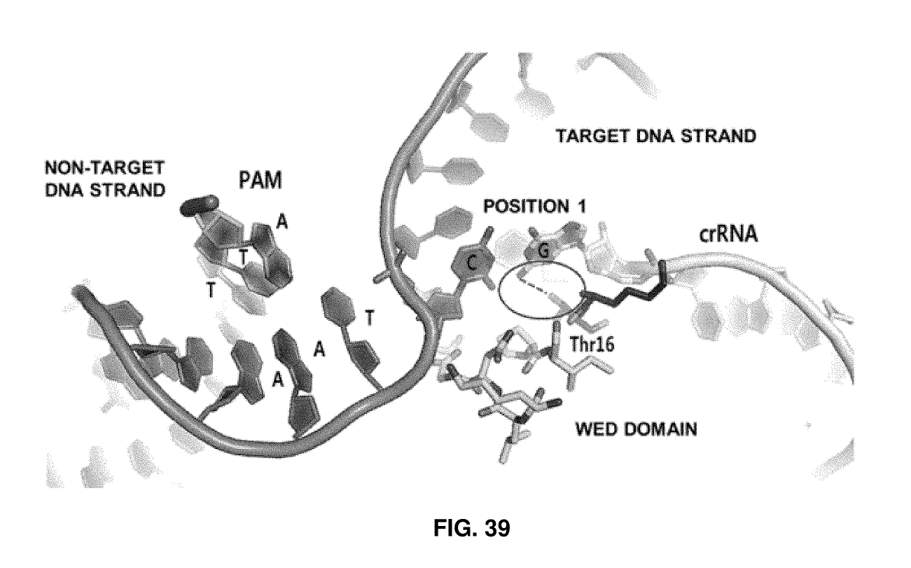

Then, the present inventors have evaluated whether the modification of a nucleotide proximal to the 5′ terminus of the TTTA PAM can affect the efficiency of genome editing. As a result, it was confirmed that there was no change in indel frequency between aTTTA, tTTTA, cTTTA, and gTTTA (FIG. 19, and FIG. 39a), whereas the indel frequency of LbCpf1 showed a high significance in a low level compared to aTTTA or tTTTA, when cTTTA was used as a PAM sequence (FIG. 39b).

### Experimental Example 1-4: High-Throughput Profiling of On-Target Activity

Then, the present inventors have attempted to confirm the characteristics of target sequences related to the efficiency of guide RNA. Considering that screening of a plurality of guide RNAs is an essential starting point in genome editing, the verification of characteristics of target sequences will be able to promote the development of genome editing technology.

First, the present inventors have evaluated whether the AsCpf1 and the Streptococcus pyogenes-derived Cas9 (SpCas9) have similar activity to the same target sequence. Considering difference between positions of PAM sequence of Cas9 and Cpf1, they have compared the activity ranking of Cas9 and Cpf1, which target both the original target sequence and the reverse target sequence (FIG. 20). As a result, it was confirmed that there is no correlation between Cas9 and Cpf1 in all cases.

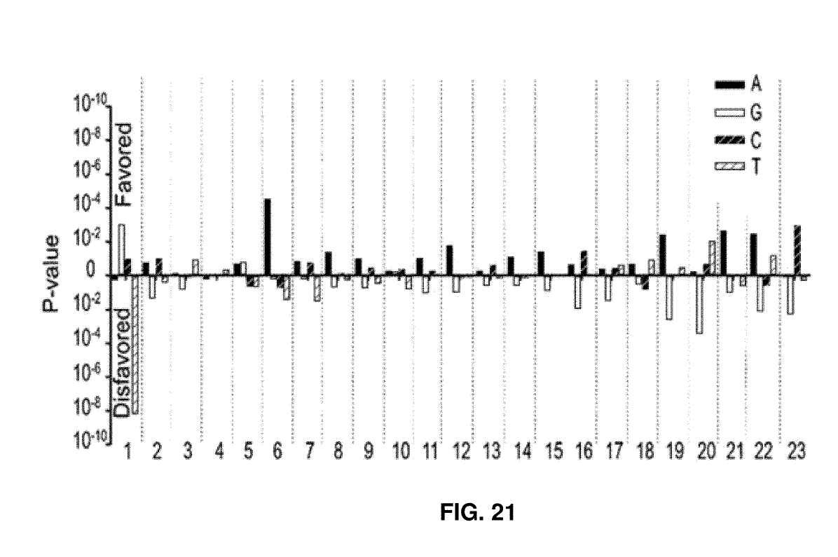

Then, the nucleotide preference of the AsCpf1 target sequence at each position was examined for 20% of guide RNAs with highest activity. The most striking difference was observed at position 1, which is the nucleotide immediately next to the PAM sequence. In the guide RNA with high activity, thymine was significantly reduced at position 1 (FIG. 21). Although there is a deviation in sequence-specific characteristics, the position immediately next to the PAM is very important in SpCas9 as well.

The present inventors have determined that the lack of preference to thymine at position 1 was due to the instability of interaction between Cpf1 protein and crRNA ribonucleotide that binds to position 1 of a target nucleotide. Based on the structure of DNA-binding AsCpf1 (PDB 5643), the hydroxy side chain of the Thr16 within the WED domain forms a stable polar interaction with the N₂ of guanine base, and also forms the same with O₂ of uracil and thymine (FIG. 39).

However, there is no corresponding moiety that can interact with the hydroxy side chain of the Thr16 in adenine, and thus the position of the crRNA adenine ribonucleotide is unstable. Therefore, the thymine at position 1 of the target DNA strand is not preferred.

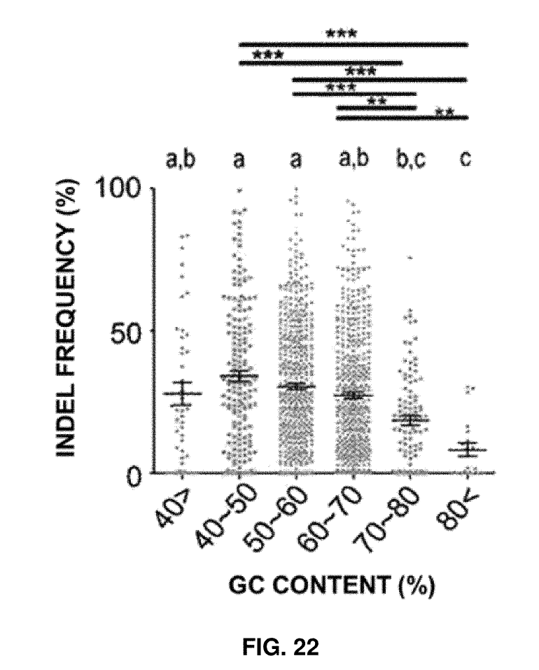

Finally, the present inventors have confirmed that AsCpf1 exhibits the highest activity with regard to a target sequence having a GC content of 40% to 60% (FIG. 22). This result is similar to the previous result with regard to SpCas9.

Indel frequency is also affected by the length of time for the expression of Cas9 and guide RNA in cells. It was reported in the previous study that when cells were subjected to a long-term culture, for example, 6 to 11 days after the transduction of the lentivirus vector that expresses Cas9 and guide RNA, the indel frequency and knock-out efficiency increase in a time-dependent manner. However, these previous studies were tested for a relatively short period (up to 14 days) with regard to only a small number of guide RNAs (1, 5, or 6), and thus, it had not been explicitly confirmed whether a long-term culture may cause an indel frequency sufficient for overcoming the limitations by sequences with regard to the guide RNA efficiency. In the screening studies at the genomic level where the indel frequency significantly affects in the screening efficiency, major nuclease (i.e., Cas9) and guide RNA are delivered to the lentivirus vector, this is a very important issue. Therefore, the present inventors have attempted to explain the above issue by the analysis of indel frequencies for the 220 guide RNAs expressed for up a month (31 days). When AsCpf1 was delivered to the lentivirus vector, the average and each indel frequency were both significantly increased by increasing the culture period to 5 days (FIGS. 23 and 24). This result is similar to the previous result with regard to SpCas9. However, 5, 10 and 31 days after transduction, the indel frequencies were no difference. These results suggest that the cultivation of 5 or more days cannot increase the indel frequency beyond a particular level, which is mainly determined by the target sequence and the guide RNA sequence.

### Experimental Example 1-4: High-Throughput Profiling of Off-Target Activity

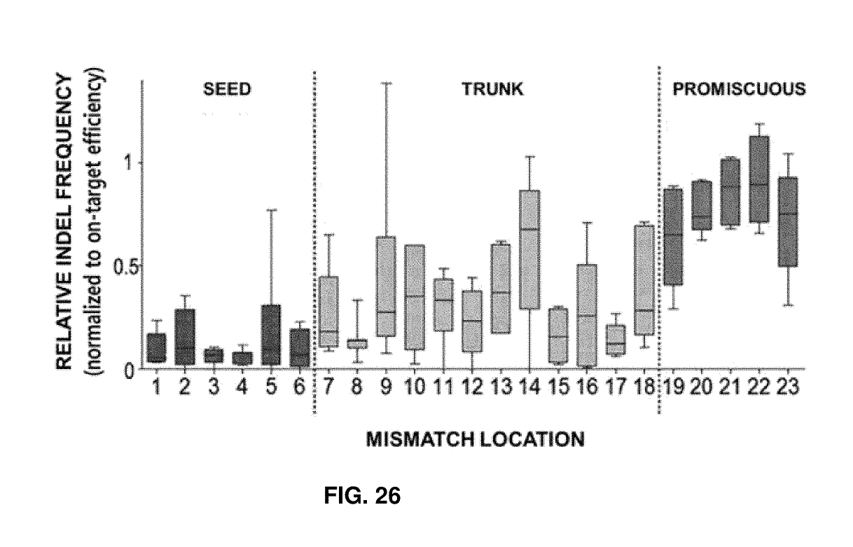

Then, the present inventors have attempted to evaluate the off-target activity profile of Cpf1. As a first step, they have attempted to confirm the mismatch effect of the guide RNA sequence with high target cleavage efficiency. In this regard, four guide RNAs for AsCpf1 and four target sequences corresponding thereto were designed, and the target indel frequencies to these were shown to be 53%, 34%, 32%, and 15% at 5 days after transduction, respectively. Among these, the three guide RNAs with the highest target cleavage efficiency were selected for off-target effect profiling, and their mismatch effects with the target sequences at each position of the guide RNAs were analyzed (FIG. 25). As a result, it was confirmed that one bp mismatch in positions from 1 to 6 significantly reduced the indel frequency (FIG. 26). These results suggest that the above positions are a seed region. As described above, the seed region of guide RNA for AsCpf1, which is verified in vivo conditions of the present invention, is similar to the results of conventional in vitro experiments where the seed region of the guide RNA with regard to the Francisella novicida-derived Cpf1 (FnCpf1) was predicted to be present within the first five positions. Meanwhile, in a case where there is a mismatch of one nucleotide sequence at positions 19 to 23, the indel frequency was shown to decrease slightly (FIG. 26). Accordingly, the present inventors have named this region as a promiscuous region.

Furthermore, in a case where there is a mismatch of one nucleotide sequence at positions 7 to 18, the indel frequency was shown to decrease moderately (FIG. 26). Accordingly, the present inventors have named this region as a trunk region.

From the above results, the present inventors have determined that, in AsCpf1, the nucleotide sequence mismatch in the seed region of the guide RNA and within the 18 nucleotides (nt) in the trunk region is intolerable, whereas the nucleotide sequence mismatch in the promiscuous region is tolerable. These results are consistent with the results of the previous studies that, in in vitro DNA cleavage of FnCpf1, it is sufficiently efficient even though the 6 nt at the 3′ terminus of guide RNA is cleaved or 18 nt of guide sequence is conserved. Additionally, even with regard to Cas9, it was previously reported that a guide RNA region located distant from a PAM sequence is not important.

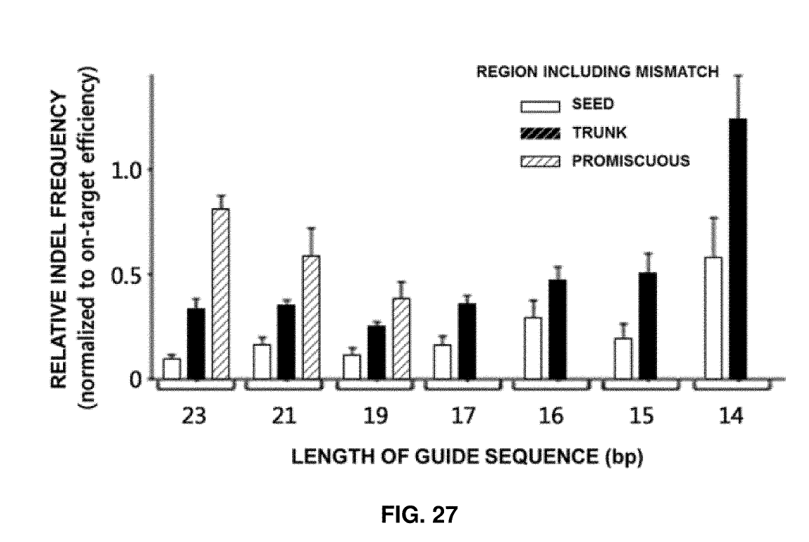

Accordingly, the present inventors then analyzed the on-target and off-target effects using a cleaved guide RNA. As a result, it was confirmed that when the 3′ terminus of a guide RNA was cut to a size of 4 nt or the length of the guide RNA was shortened to a minimum 19 nt, the on-target indel frequency was maintained and the off-target indel frequency was slowly reduced (FIG. 27). These results indicate that the off-target effect can be reduced without a decrease in on-target effect using a cut guide RNA, similar to the effect observed in SpCas9.

### Experimental Example 1-5: Library-Based Evaluation of Cpf1 Activity Having High Correlation with Indel Frequency of Endogenous Target Position

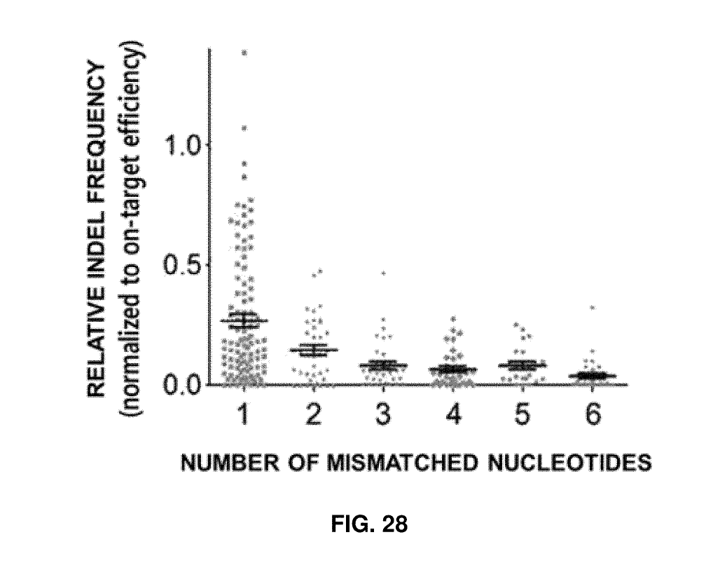

The present inventors have analyzed the correlation between the number of nucleotide mismatch and off-target effect. As a result, it was confirmed that as the number of nucleotide mismatch at a potential off-target position increased, the off-target effect reduced (FIG. 28).

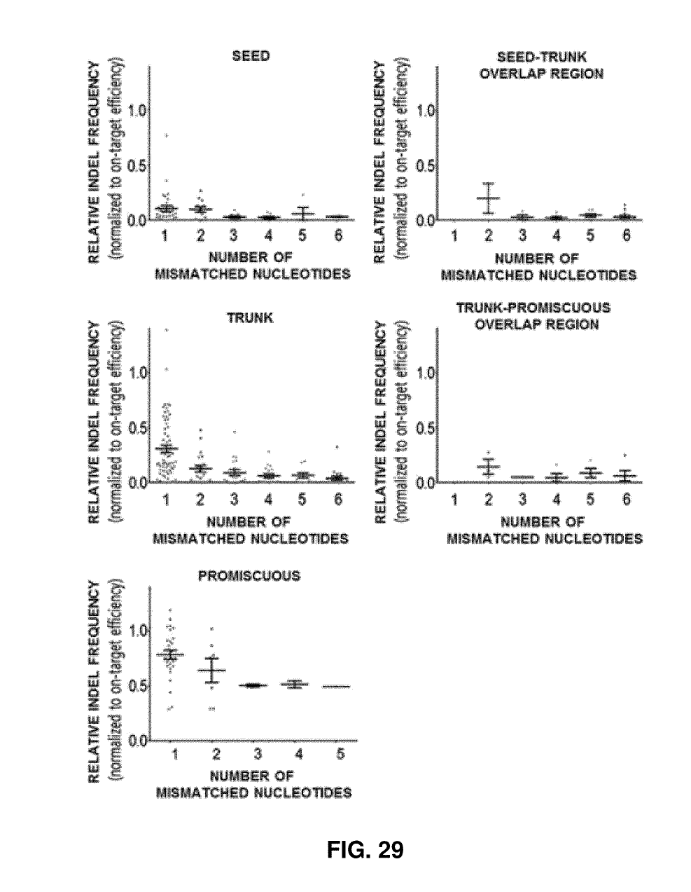

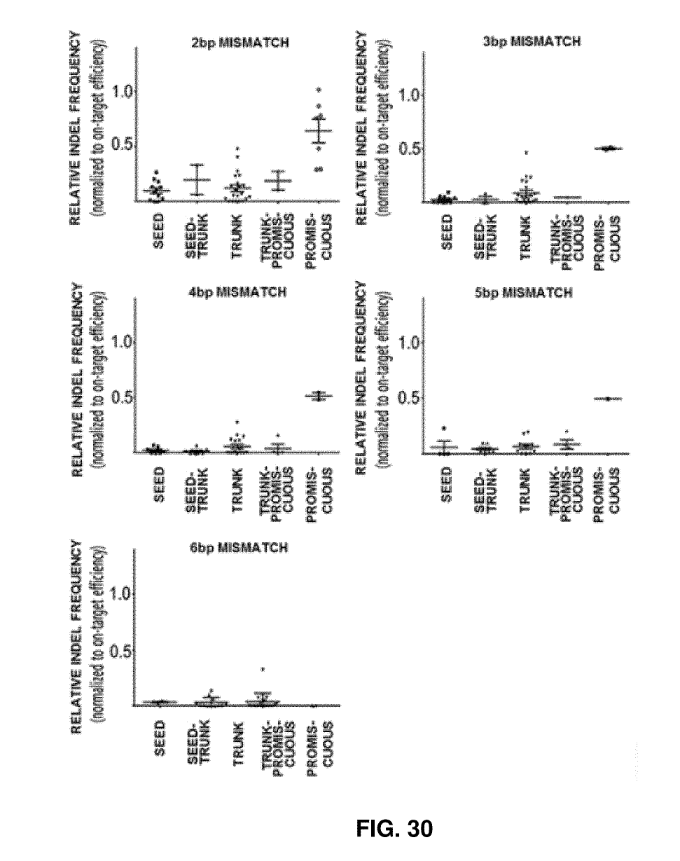

Furthermore, the present inventors have evaluated the effect of the number of nucleotide mismatch in the five regions consisting of a seed region, a region where a seed is connected to a trunk, a trunk region, a region where a trunk is connected to a promiscuous region, and a promiscuous region. As a result, it was confirmed that as the number of nucleotide mismatch increased, the indel frequency became low in all of the regions. However, in the promiscuous region where a significant indel frequency was shown even when there were 4 to 5 mismatches, this trend was not explicitly shown (FIGS. 29 and 30). Additionally, in the seed region or the region where a seed is connected to a trunk, the mismatch of 3 or more of nucleotides perfectly inhibited indel formation.

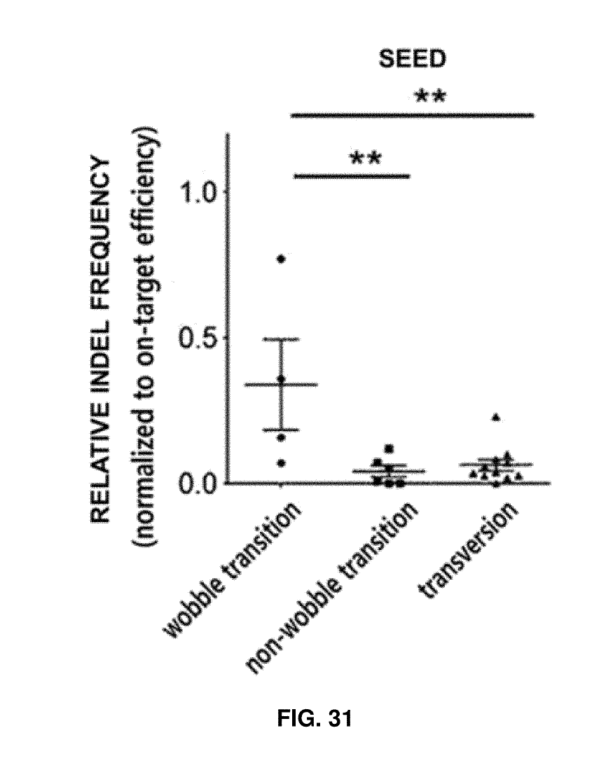

Then, the present inventors have examined whether the form of a mismatch can affect the off-target effect. In the seed region and the trunk region, it was confirmed that wobble transition mismatches were correlated with a high indel frequency, compared to non-wobble transition or transversion mismatches (FIGS. 31 to 33). These results are consistent with the unbiased analysis result with regard to the off-target effect of SpCas9. However, such a phenomenon was not observed in the promiscuous region where all types of mismatches only slightly reduced the indel frequency.

### Experimental Example 2: Evaluation of Cas9 Activity Using Pair Library

### Experimental Example 2-1: Preparation of Pair Library for Evaluation of Cas9 Activity

For the evaluation of the activity of Cas9 along with various guide RNAs in a high-throughput manner, the present inventors have prepared a guide RNA-target sequence pair library. They have amplified by PCR a pool of 89,592 array-synthesized oligonucleotides including the target sequences and the guide RNA sequences corresponding thereto (FIG. 35), and cloned with a lentivirus plasmid using the Gibson assembly (FIG. 36).

To prepare a cell library, which expresses a guide RNA and includes its corresponding sequence in the genome, the lentivirus library prepared from the plasmid library was treated on the HEK293T cells (FIG. 5).

Then, to induce the cleavage by the guide RNA and indel formation to the target sequence inserted into the genome, the Cas9-expressing lentivirus vector was transduced into cells thereby delivering Cas9 to the cell library. Then, the target sequence was amplified by PCR, and deep sequencing-based analysis was performed for the evaluation of indel frequency.

### Experimental Example 2-2: Evaluation of Cas9 Activity with Regard to Guide RNA of Human CD15 Gene and Human MED1 Gene

The Cas9 activity with regard to the guide RNA of human CD15 gene and human MED1 gene was evaluated using the pair library prepared in Examples above.

Specifically, the accuracy of the pair library was evaluated by comparing the activity ranking of the guide RNAs using the pair library and the activity ranking of the guide RNA disclosed in the literature (Nat Biotechnol, 2014, 32:1262-1267, Nat Biotechnol, 2016, 34:184-191).

As a result, the guide RNAs with regard to human CD15 gene showed the Spearman correlation coefficient of R=0.634, whereas the guide RNAs with regard to human MED1 gene (designed within top 80% of the entire length of the exon) showed the Spearman correlation coefficient of R=0.582, thus confirming that the two pair libraries have high correlation with the activity ranking of known guide RNAs (FIG. 37).

### Experimental Example 2-3: Comparison of Guide RNA Activity for Intracellular Target Sequence and Guide RNA Activity of Pair Library

The present inventors have attempted to compare the correlation between the degree of activity of the guide RNA obtained using the pair library method and the degree of activity of the guide RNA obtained by direct analysis of the target sequence present in cells.

Specifically, HEK293T cells were inoculated into a 48-well dish, and transduced with the lentivirus vector including a guide RNA-target sequence pair. 3 Days after the transduction, the cells were treated with puromycin (2 pg/mL) and only the transduced cells were selected.

Then, the SpCas9-expressing virus vector was transduced into cells in DMEM containing 10% fetal bovine serum (FBS, Gibco), and maintained in DMEM containing 10% FBS and blasticidin S (10 μg/mL, InvivoGen). After 6 days of transduction of the SpCas9-expressing virus, genomic DNA was isolated from the cell library using the Wizard Genomic DNA purification kit (Promega). Then, the target sequence inserted into the lentivirus and the target sequence present in the cell were first amplified by PCR using Phusion polymerase (NEB) for the analysis of indel frequency. For each sample, the reaction (20 μL) was performed using the genomic DNA (100 ng) per reaction. Then, the PCR products were purified using the MEGAquick-Spin™ Total Fragment DNA Purification Kit (Intron).

In the secondary PCR, the purified product of the primary PCR (20 ng) was attached along with the Illumina adaptor and a barcode sequence. The primers used in PCR reactions are shown in Table 3 below. The final products were separated, purified, and mixed, and subjected to analysis using the MiSeq or HiSeq (Illumina).

As a result, it was confirmed that the guide RNA activity for the intracellular target sequence and the guide RNA activity of the pair library were shown to have high correlation (R=0.546).

From the above result, it was confirmed that the evaluation performed in a high-throughput manner using the SpCas9 guide RNA-target sequence pair library of the present invention has high accuracy (FIG. 38).

### Experimental Example 3: Comparison with Conventional Method of Evaluating Cpf1 Activity in Target Sequence

The high-throughput method of the present invention for evaluating activity was compared to the existing individual evaluation method.

Specifically, the cost is in USD, and the unit of labor represents the maximum amount of work that can be achieved by those skilled in art for one hour. If there is a break of more than one hour, such as incubation time, it was not counted as labor.

The results are shown in Table 6 below.

TABLE 6

| | Conventional individual test | | | Method of the present invention | | |
|---|---|---|---|---|---|---|
| | | cost | labor | | cost | labor |
| Category | process | (USD) | (unit) | process | (USD) | (unit) |
| Synthesis of | synthesis | 54,000 | — | synthesis | 2,200 | — |
| oligonucleotide | | | | | | |
| Preparation of | phosphorylation | 4,480 | 100 | amplification of | 53 | 0.5 |
| library | | | | oligonucleotide | | |
| | | | | library | | |
| | ligation | 128 | 20 | Gibson assembly | 159 | 0.5 |
| | transformation | — | 220 | transformation | 165 | 1 |
| | and plating | | | and plating | | |
| | plasmid | 30,000 | 200 | plasmid | 112 | 3 |
| | preparation and | | | preparation and | | |
| | sequencing | | | cell library | | |
| | | | | preparation | | |
| Delivery of | transfection | 749 | 100 | transduction | — | 1 |
| CRISPR-Cpf1 | | | | | | |
| Preparation of | isolation of | — | 500 | isolation of | — | 2 |
| sample for deep | genomic DNA | | | genomic DNA | | |
| sequencing | PCR for deep | — | 100 | PCR for deep | — | 1 |
| | sequencing | | | sequencing | | |
| Subtotal | | 89,357 | 1,240 | | 2,689 | 9 |

To summarize the above results, the present invention provides a method for high-throughput evaluation of the activity of guide RNA with regard to a particular target sequence in a mammalian cell. It is confirmed that, for genome editing on a particular region of genome or knock-out of a particular gene, guide RNAs can be designed, and in particular, indel frequency can be confirmed by a simple delivery means such as transient transfection. However, indel frequency is not only affected by the efficiency of the guide RNA itself, but also by the transfection efficiency. Accordingly, such a method for identifying the indel frequency may not be able to stably confirm the optimal guide RNA sequence due to the deviation in transfection or delivery efficiency. In the present invention, the efficiency of 10,000 or more of guide RNAs was confirmed by one trial due to a transduction and/or transfection of a single batch with regard to a cell population, and the errors that may be induced were minimized by a deviation in delivery between different batches. A slightly lower efficiency of transduction or transfection may be able to reduce the efficiency of all of the guide RNAs tested, however, the activity ranking and “relative” activity of guide RNA are maintained, and thus it is possible to select the guide RNA with the highest activity among the tested one. One of the methods to minimize the errors that may be caused due to the different delivery efficiency is to perform repeated experiments, but this requires efforts and costs. Furthermore, the method of using a pair library of the present invention is hardly affected by epigenetic factors that variously appear according to the state and kinds of cells. Since the lentivirus vector is mostly inserted into the transcription active region, when a pair library is delivered to a cell population using the lentivirus vector, the deviation that may be induced by epigenetic state in indel frequency can be minimized. The deviations in delivery efficiency, cell state, and cell types have been raised as one of the most serious problems in comparing the efficiency of guide RNAs. However, the pair library of the present invention enables stable evaluation of the guide RNA efficiency based on sequences, and reduces the possibility that the deviation in delivery or epigenetic state may affect the efficiency.

In the case of a mid-sized unpair double library approach method that can confirm the parameters such as nucleotide sequences and epigenetic state, which may affect the activity of guide RNA, by co-transfection of about 1,400 guide RNA-encoding plasmids to cells, it is difficult to analyze off-target effect because a plurality of guide RNA libraries are co-transfected in each cell, and thus it has a disadvantage in that it is difficult to determine the confirmed indel was formed by which guide RNA. Furthermore, the copy number of the guide RNA significantly affects the cleavage efficiency, and in this case, there is a significant deviation in the copy number within a library thus making it difficult to predict the activity of each guide RNA. The library of the present invention also has a deviation in copy number similarly as in the existing libraries. However, in the present invention, the guide RNA and target sequence are used in the form of a pair, the reaction between the synthesized target sequence and the guide RNA which does not respond to its sequence can be ignored when several pairs are delivered to cells. In addition, a particular guide RNA and the DNA which encodes a synthesized target sequence corresponding thereto are present as a single copy in almost all cells, and thus the deviation associated with copy number can be prevented. Even when a similar on-target sequence is used for the evaluation of off-target, as more copy numbers are introduced than the diversity of the guide RNA sequence, the reaction between a different pair of a guide RNA and a target sequence may not appear at a significant level and thus off-target effect can be evaluated. Moreover, the number of copies to be introduced can be controlled by diluting the lentivirus vector.

The present invention enables the determination of parameters that may affect the manipulation of the RNA-guided genome. That is, the indel frequency can be confirmed at the on-target and off-target positions by various factors, such as a target sequence, kinds of effector nuclease orthologs, structural regions of guide RNA, epigenetic state of target DNA, concentration and duration being exposed to guide RNA and effector nuclease, delivery efficiency of guide RNA and effector nuclease, etc. It is expected that the effects of each parameter in various target sequences can be tested in a high-throughput manner through the pair library of the present invention.

To summarize the above results, the present invention provides a new method for detecting off-target effect. The off-target effect can be predicted through the in silico approach based on the guide-sequence similarity, and can be experimentally measured. Unbiased experimental methods, such as GUIDE-seq, Digenome-seq, BLESS, IDLV capture, HTGTS, etc. have been introduced, but they are not perfectly sensitive or elaborate.

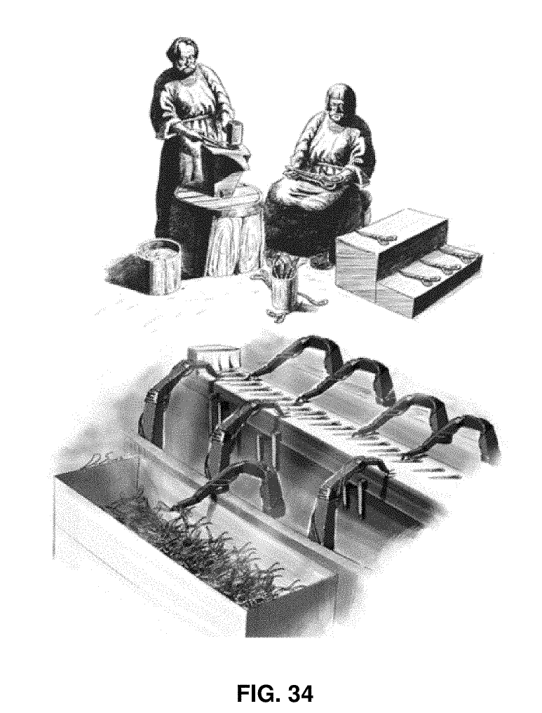

The present study may be considered as “industrial revolution” in the RNA-guided nuclease field. From now on, due to the present invention, the activity of RNA-guided nucleases can be measured in vivo in a high-throughput manner (a factory system) based on libraries, instead of relying on the conventional difficult and individual measurement system (a cottage system) (FIG. 34).

From the foregoing, a skilled person in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents, and other embodiments that may be included within the spirit and scope of the present invention as defined by the appended claims.

1. A method for evaluating the activity of an RNA-guided nuclease, comprising:
(a) performing sequence analysis using DNA obtained from a cell library, where an RNA-guided nuclease is introduced, which comprises an oligonucleotide, comprising a guide RNA-encoding nucleotide sequence and a target nucleotide sequence which the guide RNA targets; and
(b) detecting the indel frequency of each guide RNA-target sequence pair from the data obtained from the sequence analysis.

2. (canceled)

3. The method of claim 1, wherein the oligonucleotide includes a protospacer adjacent motif (PAM) sequence.

4. (canceled)

5. The method of claim 1, wherein the oligonucleotide comprises a guide RNA-encoding sequence, a barcode sequence, a PAM sequence, and a target nucleotide sequence in the 5′ to 3′ direction or in the reverse direction.

6. (canceled)

7. The method according to claim 1, wherein the oligonucleotide consists of a sequence of 100 to 200 nucleotides.

8. The method according to claim 1, wherein the guide RNA present in one oligonucleotide is cis-acting on a target nucleotide sequence present in the same oligonucleotide.

9. The method according to claim 1, wherein the method comprises:
(a) introducing an RNA-guided nuclease into a cell library, which comprises an oligonucleotide, comprising a guide RNA-encoding nucleotide sequence and a target nucleotide sequence which the guide RNA targets;
(b) performing deep sequencing using the DNA obtained from the cell library where an RNA-guided nuclease is introduced; and
(c) detecting the indel frequency of each guide RNA-target sequence pair from the data obtained from the deep sequencing.

10. The method according to claim 1, wherein the RNA-guided nuclease is a Cas9 protein or Cpf1 protein.

11. The method of claim 10, wherein the Cas9 protein is derived from at least one microorganism selected from the group consisting of the genus Streptococcus, the genus Neisseria, the genus Pasteurella, the genus Francisella, and the genus Campylobacter.

12. The method of claim 10, wherein the Cpf1 protein is derived from at least one microorganism selected from the group consisting of the genus Candidatus Paceibacter, the genus Lachnospira, the genus Butyrivibrio, the genus Peregrinibacteria, the genus Acidominococcus, the genus Porphyromonas, the genus Prevotella, the genus Francisella, the genus Candidatus Methanoplasma, and the genus Eubacterium.

13. The method according to claim 1, wherein the characteristics of the RNA-guided nuclease include at least one selected from the group consisting of:
(i) a PAM sequence of the RNA-guided nuclease;
(ii) on-target activity of the RNA-guided nuclease; or
(iii) off-target activity of the RNA-guided nuclease.

14. The method of claim 1, wherein the sequence analysis is performed by deep sequencing.

15. (canceled)

16. A vector comprising an isolated oligonucleotide, which comprises a guide RNA-encoding nucleotide sequence and a target nucleotide sequence which the guide RNA targets.

17. The vector of claim 16, wherein the vector is a virus vector.

18. (canceled)

19. A vector library comprising at least two kinds of vectors, wherein each vector is the vector of claim 16.

20. (canceled)

21. (canceled)

22. A method for constructing the oligonucleotide library, comprising:
(a) setting a target nucleotide sequence, which is to be targeted with an RNA-guided nuclease;
(b) designing a guide RNA-encoding nucleotide sequence, which forms a base pair with a complementary strand of the set target nucleotide sequence;
(c) designing an oligonucleotide, which comprises the target nucleotide sequence and a guide RNA that targets the same; and
(d) repeating steps (a) to (c) at least once,
wherein the oligonucleotide library comprises at least two isolated oligonucleotides, the isolated oligonucleotide comprises a guide RNA-encoding nucleotide sequence and a target nucleotide sequence.

23. The method of claim 22, wherein step (c) or step (d) further comprises synthesizing a designed oligonucleotide.

24.-28. (canceled)
